(19) United States
(12) Patent Application Publication
(10) Pub. No.: US 2023/0258618 A1
(43) Pub. Date: Aug. 17, 2023

(54) RAPID DETECTION OF ANALYTES

(71) Applicant: Xibus Systems, Inc., Watertown, MA (US)

(72) Inventors: Timothy Manning Swager, Newton, MA (US); Peter L. Antionette, Nashua, NH (US); Matthias Oberli, Cambridge, MA (US); Brandon Selover, Worcester, MA (US); Kent Harvey, Boston, MA (US)

(73) Assignee: Xibus Systems, Inc., Watertown, MA (US)

(21) Appl. No.: 18/160,160

(22) Filed: Jan. 26, 2023

Related U.S. Application Data
(60) Provisional application No. 63/303,554, filed on Jan. 27, 2022.

(51) Int. Cl. G01N 33/18; G01N 33/563; G01N 33/58; G01N 33/543; G01N 33/569; B01L 3/00

(52) U.S. Cl. CPC G01N 33/18; B01L 3/50; G01N 33/563; G01N 33/583; G01N 33/54346; G01N 33/56911; G01N 2469/00

(57) ABSTRACT

Rapid detection of analytes including, for example, systems, kits, and methods for growth, isolation, and/or monitoring of analytes are generally disclosed. In some embodiments, the systems and methods described herein are generally directed to the capture and/or concentrating of a target species (e.g., analyte) to be detected and/or monitored. In some embodiments, the materials, systems, and methods described herein may be used to create luminescent signals in response to the presence of selected analytes such as bacteria, viruses, and parasites. In some cases, the target analyte is a pathogenic bacteria, a pathogenic virus, a pathogenic parasite, or toxin.

### RELATED APPLICATIONS

This Application claims priority under 35 U.S.C. § 119(e) to U.S. Provisional Application Serial No. 63/303,554, filed on Jan. 27, 2022, the entire contents of which are hereby incorporated herein by reference in its entirety for all purposes.

### FIELD OF THE INVENTION

Embodiments described herein generally relate to rapid detection of analytes and related methods, kits, and systems.

### BACKGROUND OF THE INVENTION

Rapid detection of pathogenic organisms, viruses, and other biomolecular targets (e.g., analytes) is central to ensuring food/water/beverage quality and to monitor and diagnose disease. In some applications it may be important to be able to determine if pathogenic organisms are alive or dead. It is also desirable, in some cases, to create methods that are capable of detecting the target species at trace levels.

For example, pathogenic bacteria in food and beverages represents a major health concern and can cause hospitalization and even death. Food security is of increasing importance and tainted products that cause sickness in the consumers is a major liability to producers and, beyond the consequences to human health, can cause economic E. damages in terms of brand erosion. Pathogenic bacteria include Listeria monocytogenes, coli, Salmonella enterica, Legionella, Campylobacter jejuni, and Toxoplasma gondii. Viruses can also spread through food including the influenza A, influenza B, and Norovirus. Improved methods are needed to rapidly detect these pathogens in food. In some cases, such monitoring and testing, especially for pathogenic and/or toxic analytes, may require the use of expensive and complex Biosafety Level 2 facilities or higher. Accordingly, improved systems and methods are needed.

### SUMMARY OF THE INVENTION

Systems, kits, and methods for growth, isolation, and/or monitoring of analyte (e.g., pathogenic analytes) are generally provided.

In one aspect, systems for monitoring of a pathogenic analyte are provided. In some embodiments, the system comprises a reservoir configured to receive a sample suspected of including the pathogenic analyte, wherein the reservoir is configured to be essentially closed with respect to the pathogenic analyte, a capturing surface disposed within the reservoir, configured to selectively capture the pathogenic analyte, a sterile growth medium formulated to preferentially grow the pathogenic analyte disposed within the reservoir; the growth medium comprising a plurality of signaling entities comprising a moiety capable of binding to the pathogenic analyte, if present.

In another aspect, kits are provided. In some embodiments, the kit comprises a reservoir configured to receive a pathogenic analyte and a growth medium, wherein the reservoir is configured to be essentially closed with respect to the pathogenic analyte once closed, a capturing surface disposed within the reservoir, configured to selectively capture the pathogenic analyte, and a sterile growth medium formulated to preferentially grow the pathogenic analyte disposed within the reservoir; the growth medium comprising a plurality of signaling entities comprising a moiety capable of binding to the pathogenic analyte.

In yet another aspect, methods for monitoring growth of a pathogenic analyte are provided. In some embodiments, the method comprises introducing a sample suspected of comprising the pathogenic analyte into a reservoir, introducing a sterile growth medium formulated to preferentially grow the pathogenic analyte into the reservoir, closing the reservoir with respect to the pathogenic analyte, culturing, for a desired period of time, the sample, mixing the growth medium comprising the sample with a plurality signaling entities comprising a moiety capable of binding to the pathogenic analyte, if present, isolating, via a capturing surface disposed within the reservoir, the pathogenic analyte, and determining a property of the pathogenic analyte, wherein the capturing surface is configured to selectively capture the pathogenic analyte while permitting the growth medium to pass through the capturing surface.

Other advantages and novel features of the present invention will become apparent from the following detailed description of various non-limiting embodiments of the invention when considered in conjunction with the accompanying figures. In cases where the present specification and a document Incorporated by reference include conflicting and/or inconsistent disclosure, the present specification shall control

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1A is a schematic diagram of an exemplary system for monitoring an analyte, according to one set of embodiments.

FIG. 1B is a schematic diagram of an exemplary system for monitoring an analyte, according to one set of embodiments.

FIG. 1C is a schematic diagram of an exemplary system for monitoring an analyte, according to one set of embodiments.

FIG. 2A is an exemplary image of 1-3 CFU per sample of Listeria taken after 12 hours (right) compared to no listeria present on the black membrane (left), according to one set of embodiments.

FIG. 2B is an exemplary schematic illustrating pixel islands that could be detected as an image and the different shapes and sizes of the pixels and how a large dust particle can be eliminated, according to one set of embodiments.

FIG. 3 is data analysis for a bacterial assay showing the ability to differentiate between background signals (control samples, Cont 1 and 2) and three separate analyses with different ranges of island sizes, according to one set of embodiments. The factors given are the difference in average signal relative to background and each dot represents the data collected from a given image.

FIG. 4 is an exemplary image for a capture surface from an assay conducted on a control sample (no target bacteria) (304), and a successful detection wherein the diffuse features are caused by restricting the flow of sample solutions through the capture material (407), according to one set of embodiments.

FIG. 5 are exemplary images of patterns formed from the assembly of bacteria and light producing particles on a capture surface at bacterial concentrations of two controls without Listeria, two 107CFU and two 106CFU, according to one set of embodiments.

### DETAILED DESCRIPTION

Rapid detection of analytes including, for example, systems, kits, and methods for growth, isolation, and/or monitoring of analytes are generally disclosed. In some embodiments, the systems and methods described herein are generally directed to the capture and/or concentrating of a target species (e.g., analyte) to be detected and/or monitored. In some embodiments, the materials, systems, and methods described herein may be used to create luminescent signals in response to the presence of selected analytes such as bacteria, viruses, and parasites. In some embodiments, the target analyte is a pathogenic bacteria, a pathogenic virus, a pathogenic parasite, or toxin. In some embodiments, the analyte is a cell. In some embodiments, the analyte comprises a protein, a toxin, RNA, DNA, an antibody, or combinations thereof.

The phrase “pathogenic analyte” and “pathogens” as used herein is given its ordinary meaning in the art and generally refers to an analyte (e.g., bacteria, virus, parasite, fungus) that causes disease in a subject.

A “subject” refers to any animal such as a mammal (e.g., a human). Non-limiting examples of subjects include a human, a non-human primate, a cow, a horse, a pig, a sheep, a goat, a dog, a cat or a rodent such as a mouse, a rat, a hamster, a bird, a fish, or a guinea pig. Generally, the invention is directed toward use with pathogenic analytes that may cause disease in humans.

The systems and methods described herein may have numerous advantages over traditional methods for growing, isolating, and/or monitoring analytes of interest such as pathogens. For example, the systems and methods disclosed herein may, in some embodiments, have advantages in terms of simplicity, efficiency, and safety when analyzing for pathogenic or toxic biological species. In some embodiments, the systems described herein comprise a reservoir (e.g., container, vessel, channel, bag) that is advantageously configured to be essentially closed to the analyte. That is to say, in some embodiments, the analyte, once placed within the reservoir, is essentially unable to be removed from the reservoir (except for the physical and/or mechanical breakage of the reservoir). Advantageously, systems which incorporate reservoirs as described herein may permit the analysis, monitoring, and growth of pathogenic analytes without the use of expensive and complex safety equipment and/or Biosafety Level 2 (BSL2) or higher protocols and facilities. That is to say, in some embodiments, the systems and methods described do not require a biological safety cabinet or other physical containment equipment to monitor the pathogenic analyte.

In some embodiments, the use of the systems and methods described herein may advantageously provide important information about the analyte including whether the analyte is alive or dead. Advantageously, methods described are highly sensitive capable of detecting less than 100 colony forming units (CFUs) per mL rapidly, are low cost, and/or may be used with devices that enable many tests to be conducted simultaneously.

There are generally a number of possible arrangements for detection and/or determination of analytes described in this specification. In an exemplary set of embodiments, the method comprises exposing an analyte to a plurality of signaling entities (e.g., emissive particles comprising a fluorescent moiety, fluorescent particles, or the like). In some embodiments, the plurality of signaling entities comprise a moiety capable of binding to the analyte. In some embodiments, the plurality of signaling entities comprise a first moiety capable of binding to the analyte. In some embodiments, the plurality of signaling entities comprise a second moiety capable of binding to the analyte. In some embodiments, the first moiety and the second moiety are the same and/or are configured to bind to the same portion of the analyte. In some embodiments, the first moiety and the second moiety are different and/or are configured to bind to different portions of the analyte.

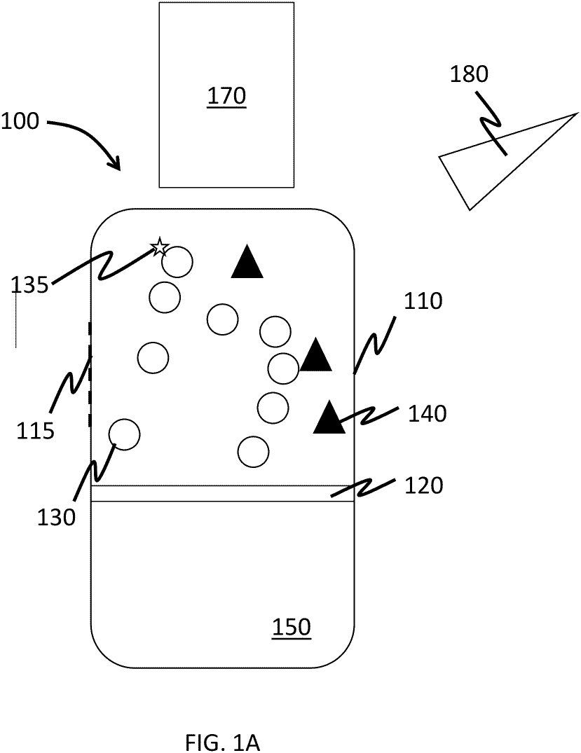

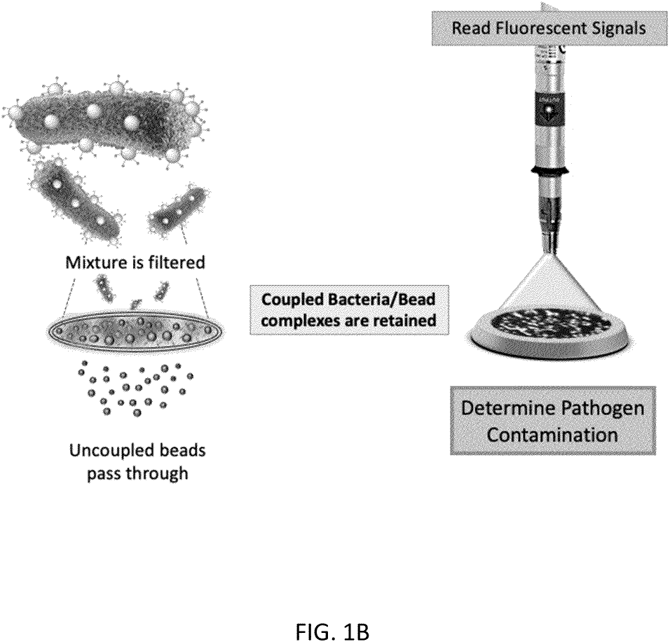

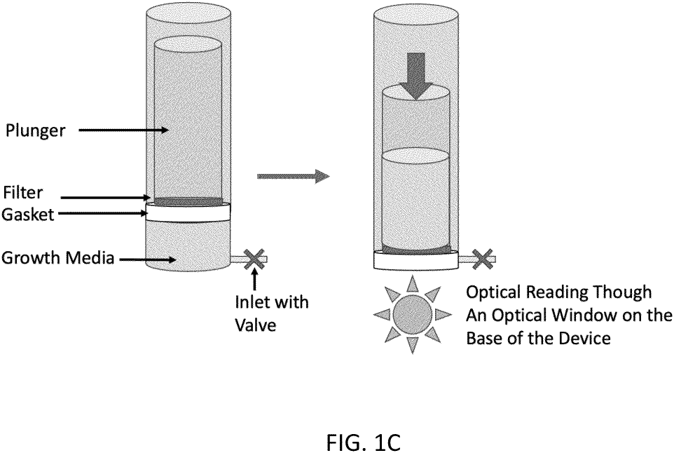

For example, as illustrated in FIG. 1A, system 100 comprises a reservoir 110. In some embodiments, reservoir 110 is configured to receive a sample (e.g., via opening 115) suspected of including a pathogenic analyte (e.g., analyte 140). In some embodiments, reservoir 110 may be essentially closed with respect to the analyte (e.g., the pathogenic analyte). For example, in some cases, once the sample is placed within reservoir 110, the analyte is unable to exit reservoir 110 (absent, for example, physical or mechanical breakage of reservoir 110). In some embodiments, after an analyte (and/or a sample comprising the analyte) is added to reservoir 110, reservoir 110 is sealed such that reservoir 110 is essentially closed with respect to the analyte (and/or the sample comprising the analyte). Non-limiting examples of suitable reservoirs include containers, vials, vessels, fluidic channels (e.g., including microfluidic channels of a microfluidic device), sample bags, or the like. Those of ordinary skill in the art would be capable of selecting, based upon the teachings of this specification, addition suitable reservoirs which may be essentially closed with respect to the analyte, as described herein.

In some embodiments, reservoir 110 is essentially closed with respect to the analyte while opening 115 permits addition of one or more materials (e.g., fluids, reagents, particles, entities, or the like) while optionally, simultaneously, preventing the passage of the analyte out of the reservoir. The opening may be in fluidic communication with one or more valves, pipes, tubes, inlets, outlets, or other fluidic components to permit the addition and/or removal of various materials (e.g., fluids, reagents, particles, entities, or the like). In some embodiments, the opening comprises a capturing surface such as a selective membrane and/or filter which can inhibit or prevent the passage of the analyte but permits the passage of one or more materials (fluids, reagents, particles, entities, or the like) that do not comprise the analyte.

“Filter,” as used herein, means a structure generally insoluble with respect to a fluid applied to it (e.g., flowed against it, flowed through it), i.e., a solid, which has the ability chemically and/or physically to inhibit the passage of at least one species associated with a fluid applied to it, relative to at least one other species. In one set of embodiments, a fluid can be positioned proximate a filter and held stationary and/or flowed relative to the filter and at least one species in the fluid can be captured at least in part by a surface of the filter either chemically and/or physically. This capture may be temporary and may impede the flow of the species relative to the fluid but not stop it, or it can be longer-term and essentially trap the species. In another set of embodiments, the filter physically inhibits and/or prevents the flow of at least one species in a fluid relative to the fluid. For example, in some embodiments, the filter is porous, and the fluid flows through the pores, where at least some pores are of a size that inhibit or prevent flow of the species. In this and other sets of embodiments, the filter preferentially separates a species of a particular average size from other species that are of smaller average size. This preferential inhibition/capture, size-dependent separation, and/or size-exclusion separation/isolation is in many embodiments different from magnetic capture of species. A filter as described herein does not necessarily require perfect conformance to the above description, but rather, the definition of a filter should be interpreted to an extent typically achievable and achieved for such structures as would be understood by those skilled in the art or as specifically described. For example, a structure may permit the passage of a relatively insignificant number of analytes, relative to the total number of analytes, to pass through the filter (e.g., due to defects, manufacturing variability, contamination, excess applied pressure, or the like), while still being understood to be a filter as described herein.

In some embodiments, the filter captures target analytes/materials at its surface. In some embodiments, the ability to capture materials may be enhanced by creating binding functionality at the surface for greater effectiveness. In some embodiments, only a selective capture surface is needed to localize materials for analysis. In some embodiments, fluid may be passed through or flowed over the capture surface.

In an exemplary set of embodiments, environmental samples collected from surfaces, from water washes, or directly from food are transferred to a detection-growth reservoir, as described herein. This reservoir may be closed, in some embodiments, in the sense that bacteria are unable to escape this system and are completely contained. The ability of the reservoir to be isolated is also useful to maintain a pre-sterilized growth media and the detection particle mixtures. In some cases, if sterility is not maintained, other bacterial can begin to grow, for example, in the reservoir prior to use. A key advantage of the isolation of samples in a detection-growth reservoir is that it will allow for testing in facilities that lack biological safety laboratories (BSL). Generally, to handle live pathogenic bacteria that are not in a sealed system a BSL-2 facility is required. As such, detection and growth containers that effectively maintain this containment may allow, in some cases, for expanded testing for food pathogens in facilities that do not have BSL-2 capabilities. Traditional pathogen detection is generally not performed at many small food production facilities for lack of the BSL-2 capabilities, and samples are usually sent out to other providers. Additionally, the lack of BSL-2 facilities often prevents food distributors from testing on site. Advantageously and illustratively, using the systems and methods disclosed herein, farmers could test for pathogenic organisms on site. The closed systems described herein provide the necessary safety for pathogen detection at sites including, for example, retail stores and restaurants, and may thereby enable broader pathogen testing than was previously possible. An expanded testing is in the public interest and will prevent sickness and deaths that occur from pathogen testing.

The detection-growth reservoir may have many different form factors and may be a flexible plastic bag, a rigid hard plastic, metal, or glass container, or combinations thereof, in addition to the examples disclosed herein. In some embodiments, the reservoir may also be fitted with equipment that allows for other materials to be injected. For example, it could have valves (e.g., associated with one or more openings such as opening 115 in FIG. 1A) that allow for material to pumped into the reservoir. Alternatively, the reservoir may comprise a septum that allows for the injection of materials through a syringe needle or the like. Bacteria samples of interest added to these detection-growth reservoirs will generally multiply and the magnetic localization methods allow for periodic measurement of the optical signals as an indication of presence of bacteria. As the bacteria grow in the reservoir, they become dangerous if released and hence a core advantage of the use of the detection and growth reservoir is that the bacteria generally never leave the reservoir as viable living organisms (e.g., except in the case of unwanted breakage of the reservoir). After monitoring the bacteria by one of more measurements over time and making a determination about the quantity of viable bacteria, the closed system may be sterilized by the addition of chemical reagents (peroxides, bleach, etc.), intense UV treatment, or thermal treatments. In some embodiments, the reservoirs are reused and in other cases the reservoirs are designed for a single use. Sterilized devices may be discarded. In some embodiments, the sterilization and disposal may be accomplished by incineration of the device. In the latter, the materials may be chosen to make this process as environmentally friendly as possible.

As described herein, the detection-growth reservoir may be initially charged with a sterile media that is selected to promote the growth of selected bacteria and optionally to suppress the growth of other bacteria. It is also possible that this mixture may be fortified over time by injections of additional media or gas. In some embodiments, the detection-growth reservoir is completely hermetically sealed. No gas, liquid or other matter is introduced or withdrawn after sample introduction until after the camber is sterilized or destroyed, in some embodiments.

Referring again to FIG. 1A, in some embodiments, system 100 comprises a capturing surface 120 (e.g., filter, or other surface) that preferentially retains an analyte 140, if present and/or preferentially retains a plurality of signaling entities 130, if present. Signaling entities are described in more detail, below. In some embodiments, capturing surface 120 is a semipermeable membrane (e.g., a filtration layer) that will allow for the transfer of gas or liquids, but will not allow bacteria to pass. For example, in some embodiments, the semipermeable membrane comprise a filter having small pore sizes of 0.2 microns and/or has hydrophobic characteristics that prevent bacteria transport. In some embodiments, the capturing surface captures the analyte and/or signaling entities via size preferential separation and/or antibody capture on the capturing surface. For example, in some embodiments, the capturing surface comprises a porosity sufficient to permit flow of a fluid while preventing passage of the analyte (e.g., the analyte bound to a plurality of signaling entities).

In some embodiments, capturing surface 120 comprises one or more moieties capable of binding to the analyte and/or the plurality signaling entities. For example, in some embodiments, one or more moieties capable of binding the analyte and/or the plurality of signaling entities may be immobilized on a surface of the capturing surface (e.g., on a surface of a filter) and/or may be present within the capturing surface (e.g., within the pores of a filter).

In some embodiments, the ability of gas or solutions to be added or withdrawn from the detection-growth reservoir may assist in the detection of bacteria. This could include simply concentrating the bacteria, but could also involve adding gas or nutrients that enhance the growth of the target bacteria and/or suppress the growth of others. For example, if the atmosphere is kept as oxygen free, only anerobic bacteria (c.a. Campylobacter) are expected to grow.

It is also possible that the detection-growth system may have multiple reservoirs, in some cases. In an exemplary embodiment, the sample is initially inserted into a reservoir and isolated. This reservoir may have a solution that extracts the bacteria and then distributes the extract into one or more other reservoirs that contain growth media that is designed to promote the growth of one target bacteria over the others. The one or more other reservoirs may also contain particles for optical signaling that are specific to an analyte of class of analytes. In this way a single sample may be analyzed for multiple bacteria and/or other analytes. The distribution to the multiple isolated detection-growth reservoirs could be done in parallel or in series. In the latter, in some embodiments, the sample can be first grown in one medium that promotes the growth a specific pathogen and then a portion of that growth mixture can be transferred to another reservoir that favors the growth of one or more other pathogens.

In some embodiments, once the sample is collected and placed in the reservoir the reservoir does not comprise an opening. In some embodiments, the reservoir is effectively sealed (e.g., hermetically sealed) such that all added materials including, for example, one or more of the sample suspected of including the analyte, the isolation particles, the signaling entities, growth media, etc. are not readily removable from the reservoir (e.g., absent physical or mechanical breakage of the reservoir). Sealing in such a way as to prevent gas diffusion into a system may be advantageous, in some embodiments, for the selective growth and detection of anerobic bacteria.

Referring again to FIG. 1A, in some embodiments, a plurality of signaling entities 130 (e.g., fluorescent particles) may, optionally, be added to reservoir 110. In some embodiments, the plurality of signaling entities are added to the sample prior to adding the sample to the reservoir. In some embodiments, the plurality of signaling entities are added directly to the reservoir (e.g., before adding the sample, after adding the sample).

In some embodiments, plurality of signaling entities 130 are configured to bind to analyte 140, if present. In some embodiments, plurality of signaling entities 130 comprise a moiety 135 capable of binding with analyte 140, if present.

Signaling entities are known in the art, and those of ordinary skill in the art would be capable of selecting suitable signaling entities to be used in the invention based upon the teachings of this specification. In a subset of embodiments, signaling entities described below are used. Signaling entities may be particulate or non-particulate. Where non-particulate, they may be chemical (small molecule or polymers) or biological species. Particulate signaling entities may define particles which themselves may emit detectable signals through emission or the like, or may be functionalized on one or more surfaces with such signaling entities. For example, a signaling particle may have a surface or interior functionalized with signaling entities which signal when exposed to a source of excitation energy described herein, and may also be functionalized with linking moieties to link to an analyte.

In some embodiments, the signaling entity is an emissive particle. In some embodiments, the signaling entity is a recognition element (i.e. moiety) that is emissive. In some embodiments, the signaling entity is an emissive particle containing one or more recognition elements. In some embodiments, the signaling entity is an emissive polymer containing one or more recognition elements. In some embodiments, the signaling entity contains an emissive species containing Eu, Tb, Gd, Au, Au, Ir, Cu, Pd, Pt, Ru, Ag, Zn, or Al. In some embodiments, the signaling entity is an emissive polymer is a polyfluorene containing one or more recognition elements. In some embodiments, the signaling entity is an emissive polymer capable of transferring energy to a minority chromophore that one or more recognition elements.

In an exemplary set of embodiments, the plurality of signaling entities comprise a receptor-dye conjugate (e.g., comprising an antibody or recognition protein) and/or a nanoparticle (e.g., an emissive nanoparticle such as a light-producing nanoparticle). Other signaling entities are also possible and are described in more detail, below. In some embodiments, the signaling entity is selected to have an excited state lifetime of greater than or equal to 1 microsecond (e.g., greater than or equal to 2 microseconds, greater than or equal to 5 microseconds, greater than or equal to 10 microseconds). In some embodiments, that signaling entity is selected to have an excited state lifetime less than 1 microsecond, less than 100 nanoseconds, or less than 10 nanoseconds. In some embodiments, the signaling entity is configured to scatter light.

In some embodiments, a moiety interacts with the analyte (e.g., pathogenic analyte) via formation of a bond, such as an ionic bond, a covalent bond, a hydrogen bond, Van der Waals interactions, and the like. The covalent bond may be, for example, carbon-carbon, carbon-oxygen, oxygen-silicon, sulfur-sulfur, carbon-sulfur, phosphorus-nitrogen, carbon-nitrogen, metal-oxygen, or other covalent bonds. The hydrogen bond may be, for example, between hydroxyl, amine, carboxyl, thiol, and/or similar functional groups. For example, the moiety may include a functional group, such as a thiol, aldehyde, ester, carboxylic acid, hydroxyl, and the like, wherein the functional group forms a bond with (a portion of) the analyte. In some cases, the moiety may be an electron-rich or electron-poor moiety wherein interaction between the moiety and the analyte comprises an electrostatic interaction.

In some cases, the moiety may comprise a biological or a chemical group capable of binding another biological or chemical molecule. For example, the moiety may include a functional group, such as an azide, boronic acid, a reactive ester, a thiol, aldehyde, ester, carboxylic acid, hydroxyl, and the like, wherein the functional group forms a bond with (a portion of) the analyte.

In some embodiments, moiety and the analyte interact via a binding event between pairs of biological molecules including proteins, nucleic acids, glycoproteins, carbohydrates, hormones, and the like. Specific examples include an antibody/peptide pair, an antibody/antigen pair, an antibody fragment/antigen pair, an antibody/antigen fragment pair, an antibody fragment/antigen fragment pair, an antibody/hapten pair, an enzyme/substrate pair, an enzyme/inhibitor pair, an enzyme/cofactor pair, a protein/substrate pair, a nucleic acid/nucleic acid pair, a protein/nucleic acid pair, a peptide/peptide pair, a protein/protein pair, a small molecule/protein pair, a glutathione/GST pair, an anti-GFP/GFP fusion protein pair, a Myc/Max pair, a maltose/maltose binding protein pair, a carbohydrate/protein pair, a carbohydrate derivative/protein pair, a metal binding tag/metal/chelate, a peptide tag/metal ion-metal chelate pair, a peptide/NTA pair, a lectin/carbohydrate pair, a receptor/hormone pair, a receptor/effector pair, a complementary nucleic acid/nucleic acid pair, a ligand/cell surface receptor pair, a virus/ligand pair, a Protein A/antibody pair, a Protein G/antibody pair, a Protein L/antibody pair, an Fc receptor/antibody pair, a biotin/avidin pair, a biotin/streptavidin pair, a drug/target pair, a zinc finger/nucleic acid pair, a small molecule/peptide pair, a small molecule/protein pair, a small molecule/target pair, a carbohydrate/protein pair such as maltose/MBP (maltose binding protein), a small molecule/target pair, or a metal ion/chelating agent pair. Specific non-limiting examples of moieties include peptides, proteins, DNA, RNA, PNA. Other moieties and binding pairs are also possible.

In some embodiments, a detector (e.g., a fiber optic cable, a sensor) 170 capable of detecting electromagnetic radiation is positioned proximate reservoir 110. In some embodiments, a source of electromagnetic radiation 180 is positioned proximate the reservoir (e.g., to provide an excitation that causes the signaling entities to generate detectable electromagnetic radiation).

In an exemplary set of embodiments, a property of an analyte may be determined by applying a force and/or moving capturing surface 120 such that the analyte and the signaling entities bound to the analyte are drawn towards detector 170, and the detector is configured to detect a signal from the plurality of signaling entities (e.g., also bound to the analyte), optionally in the presence of applied electromagnetic radiation. In some embodiments, the signal may be correlated with the property of the analyte.

In some embodiments, the property of the analyte includes viability (e.g., alive, dead), presence of the analyte in the sample, a growth rate of the analyte, and/or a relative amount of analyte. In an exemplary set of embodiments, repeated measurements and the use of specific receptors enable the determination if bacteria are alive or dead.

In some embodiments, the optical excitation produces an emission that can be captured as an image and analyzed. The image may be captured with the use of magnifying lenses and/or multiple images may be used in an analysis, in some embodiments. This analysis may, in some cases, include the shape, size, intensity, and color of the emission image. This image data may be used to discern signals from non-specific binding of signaling groups to non-analyte particles and thereby eliminate false positive signals.

In some embodiments, a growth medium (e.g., a sterile grown medium) 150 may be added to reservoir 110. In some embodiments, the growth medium is selected to preferentially grow the analyte of interest (e.g., the pathogenic analyte) disposed within the reservoir. One of ordinary skill in the art would be capable of selecting, based upon the teachings of this specification, growth medium which may preferentially grow an analyte of interest such as a pathogenic analyte. In some embodiments, the growth medium may permit the growth of the analyte of interest while inhibiting, killing, or otherwise growing at a substantively slower rate, other analytes such as other pathogenic analytes (e.g., pathogenic bacteria, pathogenic virus) and/or non-pathogenic analytes (e.g., non-harmful bacteria) that are not of interest.

In an illustrative embodiment, the (sterile) growth medium permits the growth of a pathogenic bacteria of interest while no other pathogenic bacteria and non-pathogenic bacteria is permitted to grow, such that, over time, the pathogenic bacteria of interest becomes the dominant species in the sample. Advantageously, the use of such selective growth medium may permit the long-term monitoring of pathogenic bacterial growth. In some embodiments, the systems and methods described herein may be used to determine if the pathogenic bacteria present in the sample is alive or dead.

The term “alive” generally refers to an analyte (e.g., bacteria) that, given optimal conditions in which the analyte (e.g., bacteria) is expected to grow, the analyte (e.g., bacteria) will multiply. In some cases, depending on the state of the analyte when collected in a sample, this process may be delayed while the analyte in effect heal over the course of a few hours. By way of example and for illustrative purposes only, if the target pathogenic bacteria does not grow in the growth medium that otherwise preferentially permits the growth of the pathogenic bacteria, the pathogenic bacteria may be considered non-viable or dead.

Non-limiting examples of pathogenic analytes with which the systems and methods may be used to monitor, grow, and/or detect the presence of include Achromobacter xylosoxidans, Acinetobacter baumannii, Actinomyces (e.g., Actinomyces israelii), Aeromonas species, Bacillus species, Bacteroides fragilis, Bacteroides melaninogenicus, Bartonella species, Bordetella pertussis, Borrelia species, Brucella species, Burkholderia species, Campylobacter, Capnocytophaga species, Chlamydophila pneumoniae, Chlamydophila psittaci, Citrobacter species, Clostridium species, Corynebacterium species, Coxiella burnetiid, Ehrlichia species, Eikenella corrodens, Enterobacter species, Enterococcus faecalis,Enterococcus faecium, Escherichia coli, Francisella tularensis, Fusobacterium necrophorum, Gardnerella vaginalis, Haemophilus species, Helicobacter Pylori, Influenza (e.g., influenza A, influenza B), Klebsiella species, Lactobacillus species, Legionella species, Leptospira species, Listeria monocytogenes, Moraxella, catarrhalis, Morganella species, Mycoplasma pneumonia, Neisseria species, Nocardia species, Norovirus, Pasteurella multocida, Peptostreptococcus species, Porphyromonas gingivalis, Propionibacterium acnes, Proteus species, Providencia species, Pseudomonas aeruginosa, Salmonella species, Serratia marcescens, Shigella species, Staphepidermidis, Staph hominis, Staph. Haemolyticus, Staphylococcus aureus, Staphylococcus saprophyticus, Stenotrophomonas maltophilia, Streptococcus agalactiae, Streptococcus anginosus group, Streptococcus pneumoniae, Streptococcus pyogenes (Groups A, B, C, G, F), Toxoplasma gondii, Treponema pallidum, and Vibrio species.

In an exemplary set of embodiments, the analyte of interest may be selected from the group consisting of Listeria monocytogenes, E.coli, Salmonella enterica, Legionella, Campylobacter jejuni, Toxoplasma gondii, influenza A, influenza B, and Norovirus.

Other analytes are also possible and those of ordinary skill in the art would be capable of selecting suitable target analytes based upon the teachings of this specification.

The systems and methods described herein may be used in a wide variety of applications in which detection of pathogenic analytes are important. For example, detecting organisms in water is also of interest and, in some embodiments, it may be ideal to detect classes or bacteria, and/or total bacteria, rather than individual bacteria. In this way total bacterial counts can be deduced. Total bacterial counts may use recognition elements (e.g., moiety) able to bind to a wide range of species.

In some embodiments, detecting and tracking of the outbreak of human disease using efficient detection methods of viruses is important to public health. The methods in this disclosure are also relevant to the detection of viruses and antibodies that can inform if an individual has previously contracted a disease.

Sensing of biological species generally uses mechanisms for their selective recognition and the ability to generate a readable signal associated with the recognition process. In general, an optical excitation is applied to a material and an emissive signal may be detected. Emissive responses, or emission, represents all photons that are the result of luminescence, scattering, or reflection. The wavelength or color of the emission may also contain information and luminescence may generate selected ranges of light. Scattered light may have different ranges of wavelengths as a result of the particle size or because of subtractive color. Reflectivity may similarly have different intensities as a function of wavelength. In the case of a reflection that angle of the reflective surface of periodic structure with regard to the direction of the excitation and direction at which the light is collected may influence the wavelength intensity distribution of the emissive signal.

There are multiple ways to generate optical signals using luminescent probes that have been widely used to study biological systems. Methods that create robust detection schemes capable of automation or with minimal user training may also benefit from the use of luminescent methods. An advantage of a luminescent method is that it may provide high sensitivity, if background signals may be eliminated. Background signals may include native fluorescence from the biological materials, media, containers, contaminates, or stray light. There are different approaches to reduce or eliminate background that may include using filters to prevent the excitation of emissive species, or to prevent the light from unwanted emissions to reach the photodetectors. Delayed luminescent probes and time-gated emission detection may also be used to collect signals from particular probes at times after background emission has subsided.

Localization of emissive species in a place wherein they would not be present without a biological signal is described in this disclosure as an additional method for the creation of detectable emission signals and low background in the absence of the biological target. Specifically, by creating a measurement method wherein emissive species may only persistently associate at a location in the presence of a biological target signal may be used to produce a robust and sensitive signal. Optimal systems optimally making these types of measurements will take into consideration methods to reduce or eliminate scattered, stray light, and interference from other emissive sources. Hence, filters, directional detection, waveguiding, and focusing optics may be used to produce a variety of systems suitable for different applications. In some embodiments, the measurement may be made inside of a sample bag. For example, in some cases a sample to be analyzed will be placed inside of a bag that will be mechanically stimulated to affect the release of bacteria. The sample may be food, tissue, plants, or a device such as a sponge that was used to collect bacterial from surfaces. Devices that provide this mechanical stimulation include what are known as stomachers and may be used for analysis for bacterial associated with food production and quality control. If the biological target analyte may cause localization of emissive species at a predetermined location, detection may be enabled without transferring the solution to another vessel. Such a process has advantages in terms of simplicity, efficiency, and safety when analyzing for pathogenic or toxic biological species. In other cases, it may be advantageous to transfer material from a bag after mechanical stimulation to liberate biological target analytes, into one or more additional vessels for analysis. The measurement within this new vessel may also make use of the ability the biological analyte to localize an emissive species at a location for measurement. Multiple vessels may be used to create specific tests for different species or to optimally extract information such as if pathogenic organisms are alive or dead, as well as provide different dynamic ranges of sensitivity. For example, vessels may be configured to provide ultralow limits of detection in a very short time, while other vessels may be measured periodically to monitor the growth of bacteria. The methods described in this disclosure may be used to detect if bacteria are alive or dead and confirm if, for example, that food processing equipment has been properly sterilized. In some embodiments, the total amount of live and dead organisms will be of interest, and in other embodiments, the detection of live bacterial will be the preferred output of the analysis. The disclosed methods may, in some embodiments, selectively detect live bacteria.

As described above, the disclosed methods make use, in some embodiments, of signaling entities functionalized with recognition elements (e.g., moieties). Non-limiting examples of the recognition elements are antibodies, proteins, oligonucleotides, DNA, RNA carbohydrates, glycoproteins, lectins, or synthetic receptors may be used to bind to biological species of interest. These particles may be released on a growth medium and after culture the number of colonies may be counted to give a measure of the bacterial. These studies are slow and generally require visual detection of colonies. They do have the advantage of differentiating bacteria that are alive from those that are dead, as only live bacterial will generally grow in culture, in some embodiments. More efficient methods to monitor the growth of bacteria in real-time, with selective detection thereof, may provide considerable benefit to society.

The methods in this disclosure are ideally suited, in some embodiments, to detect biological species (analytes) that have what is known as multivalency. The term, multivalency refers to the fact that there are multiple sites that may be binding sites upon which receptors, ligands, or other recognition species may associate with the biological analytes. If there is only one binding/interaction site that interacts with a specific receptor/recognition element, then the interaction is said to be monovalent. If there are two or more binding sites for a specific receptor then the interaction is said to be multivalent. The interacting sites may be the same or different. Hence, although there may only be a monovalent interaction with one receptor, a multivalent interaction may occur if there are one or more other receptors capable of interacting with different sites on the biological species. Cells, bacteria, and viruses are generally capable of multivalent interactions. Many proteins are also capable of multivalent interactions, either by virtue of their large size that presents multiple independent epitopes (binding sites), or because they are associated in a multimer for binding of recognition elements. An example of a relevant multimers are Shiga toxins, which are highly toxic proteins that may be secreted by certain Escherichia coli (E.coli) serotypes. The B subunits of these toxins of are pentamers and hence provide multivalency. Additionally, it is possible that multiple recognition elements may be added and that one could also bind the A subunits of a Shiga toxin and expand the multivalency. There are advantages to having multiple recognition elements in a detection system as it may provide for more robust recognition and differentiation between closely related species. For example, the disease COVID-19 is caused by a specific coronavirus, but there are other known coronaviruses. Methods that selectively detect the coronavirus responsible for COVID-19 may be accomplished by using recognition elements that selectively recognize epitopes associated with the target virus analyte. Using multiple recognition elements could be used to detect proteins that form heterodimers, by recognizing epitopes on each of the proteins involved in the complex. It is also possible that a single protein may be simultaneously bound to a ligand for which the protein is a receptor and a receptor that recognized and epitope on the protein.

Viruses, such as a coronavirus have a protein shells that provide structure and function necessary to invade host cells. These protein shells have many copies of given proteins that assemble into structures, including the capsid that surrounds and protects the viral genome. The multiple capsid proteins and viral coat proteins all provide potential recognition sites for binding. In the case of a virus, there could be a specific epitope, on each of the capsid proteins. There could in also be multiple recognition sites, on each capsid protein. Similarly, the viral coat proteins may present multiple epitopes. As a result, if a recognition group such as an antibody or engineered protein receptor that is specific to one of the epitopes is added, it is possible for it to bind to multiple sites.

It is also possible that virus particles may be detected by their RNA. There are variety of ways that assemblies of RNA or DNA may be assembled and interact with complementary recognition elements. Those who are skilled in the art will recognize that RNA or DNA may be recognized by binding of a complementary oligonucleotide, DNA or RNA. It is possible in some embodiments, to bind to multiple segments a specific RNA, DNA, or oligonucleotide by designing more than one complementary oligonucleotide, DNA or RNA binding unit.

In some embodiments, the biological detection methods described in this disclosure are enabled by the use of particles that contain luminescent, or reflective properties or combinations thereof. The particles are functionalized with recognition elements (receptors, ligands, nucleotides, or the like) that bind a biological species of interest. In some embodiments, this optical signature may be intrinsic to the analyte and in others an optical signature may be added. A non-limiting example of the later would be to add fluorescent recognition element, such as a fluorescently labeled antibody that binds to the analyte. There are a diversity of materials and recognition elements that may be used with this method.

In some embodiments, different emissive dyes with different emission colors (wavelengths) may be used to identify particles having different recognition elements. In this way, a multiplexed biological assay may be created that provides for the ability to obtain more information about the biological analytes. It is also possible to use particles capable of reflecting light. In this case interference effects within the particles may selectively reflect light of given wavelengths. Particles having multiple components with different refractive indexes and interfaces may be used to produce reflections. These include assembled block copolymers, chiral nematic liquid crystals, and multiphase colloid particles. To create highly robust methods there may be advantages, in some embodiments, to having these materials in a solid or polymer stabilized form. For example, a chiral nematic liquid crystal is technically a liquid, however the helical photonic structure of these materials may be preserved after polymerization of some or most of the molecules constituting the liquid crystal. In so doing a reflective solid or semi-solid (gel) particle may be produced.

The emissive particles may range from functionalized polystyrene particles that 10 nm to 300 microns in diameter. In some embodiments, particle like constructions may be used wherein a receptor is covalently modified with one or more molecular of polymeric emissive dyes. The latter may not be conventionally considered as a particle, but may provide the same function of a particle and one who is skilled in the art will recognize that this approach may provide the same emissive labeling to the analyte as a particle. In some embodiments polymeric dyes may provide for efficient optical absorption and emission properties. In some embodiments, these polymeric dyes may be a water-soluble conjugated polymer. Non-limiting examples of water-soluble dyes are certain polyfluorenes, polyfluorene copolymers, and polyfluorenes that have other dyes attached to them. Conjugated polymers may be used as efficient antennas for the absorption of light and transfer the energy to minority sites in the polymer or pendant dyes to create longer wavelength emission, than would have been expected from the polyfluorene backbone. In some embodiments, the emissive particles will be designed to scatter or reflect light.

In some embodiments, emissive particles may be produced by the assembly of non-water-soluble materials by different dispersion methods. For example, adding a tetrahydrofuran solution of dye-polymer mixture to water that is being vigorously stirred, vortexed, and/or being stimulated ultrasonically may produce a dispersion. For some embodiments, the materials undergoing dispersion will benefit from the addition of surfactants that stabilize the dispersions in water. The surfactants may be small molecules, particles, biomolecules, or polymers. The particles may be solids composed materials that are in a crystalline, glassy, amorphous, rubbery, plastic, or combinations thereof. In some embodiments, these phases may be interspersed with fluid phases internal to the particles. In other embodiments, the particles are fluids. A fluid particle may allow for dynamic assemblies of the surfactants and other functionality associated with their surfaces. In some embodiments, the ability to cluster recognition elements around the binding of an analyte will be desirable. Clustering of recognition elements may be used to enhance the strength of the association of the particle with the analyte and may also be used to create a specific optical signature. In some embodiments, clustering may enhance energy transfer processes that may provide a new enhanced optical signature associated with analyte binding. Particles may have clear domains and structure. In some embodiments, particles will have what is known as a Janus structure wherein there are in effect two separate domains that define two different faces to the particles. The different phases may be solids or liquids and may have different optical elements. In some embodiments, each phase will have a different luminescent dye. In some embodiments, at least one of the phases will have a dye that absorbs light and is largely non-emissive.

In some embodiments, microfluidic methods may also be used to make precision particles containing dyes or have multiple intraparticle phases encoding different optical information. In some embodiments, these particles will range in size from 1 micron to 300 microns. In some embodiments, there may be two phases within the particle that have emissive optical elements. In some embodiments one of these phases may be a polymer and one phase may be a liquid. In other embodiments, both phases may be polymers, and in yet other embodiments both phases may be liquids. Block copolymers capable of assembling into structures capable of reflecting light may similarly be dispersed to produce particles. In some embodiments, the polymers may be the dyes and in preferred structures the polymer is a conjugated polymer. A base polymer may be chosen to very efficiently absorb light and produce luminescence. In some embodiments, a secondary and minority chromophore may be incorporated either covalently or non-covalently into the polymer. If the minority chromophore is emissive and capable of accepting energy from the base polymer, then the emission wavelength may be shifted. A general requirement for such a shift in wavelength is that the emission associated with the addition of the chromophore may, in some cases, be longer wavelength (lower energy) than the emission of the chromophore donating the energy. The degree to which the emission shifts to lower energy or longer wavelength may vary. The mechanisms upon which energy may transfer between chromophores may involve dipolar coupled processes, known as Förster energy transfer or mechanisms that provide for strong electronic coupling including what is often referred to as Dexter energy transfer.

In some embodiments, the core of a luminescent particles may be substantially, or completely, composed of molecular or polymeric chromophores. For example, the π-conjugated polymers below may be dispersed in water with surfactants to create stable luminescent particles. An advantage of these materials is that they may have broad absorbances allowing for efficient excitation by a variety of light sources. The “R” groups in these structures may be independently varied and are known to one skilled in the art as indicating that a wide variety of organic groups may be attached to the structures, included by not limited to H, alkyl groups or aromatic groups. Alternatively, R may be a halogen in the case that it is bound to a carbon atom. In some embodiments, some of the R groups will be hydrophobic and some will be hydrophilic. In some embodiments, the polymers may behave as their own surfactants. In some embodiments, some of the R groups may contain reactive elements including, but not limited to, amines, azeides, carboxylates, tetrazines, trans-cyclooctenes, cyclic alkynes, maleimides, thiols, disulfides, reactive esters, or norborenes. In other embodiments, the conjugated polymers are part of a macromolecular structure known as a block or brush polymer. In a block polymer, a polymer has multiple sequences and the conjugated polymers may be flanked on one or both ends by a block polymer that may provide surfactant characteristics and/or reactive elements. In some embodiments block copolymers structures may be used to stabilize the aqueous dispersions of the conjugated polymers. In some embodiments, the non-conjugated block of the block copolymer structure has additional dyes. In some embodiments, the non-conjugated block of the block copolymer structure has hydrophilic character. In some embodiments, the non-conjugated block of the block copolymer structure has reactive groups including, but not limited to, amines, azides, carboxylates, tetrazines, trans-cyclooctenes, cyclic alkynes, maleimides, thiols, disulfides, reactive esters, or norborenes.

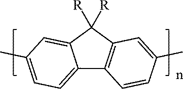

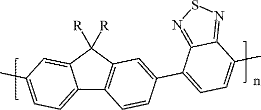

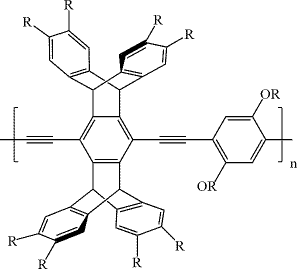

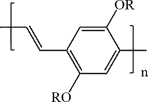

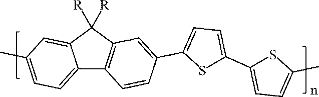

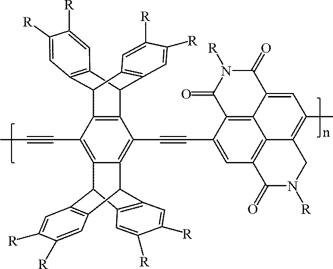

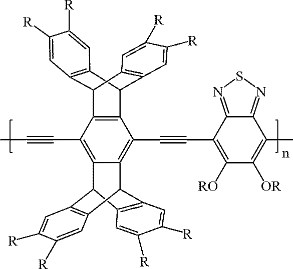

Conjugated π-conjugated polymers are generally efficient at energy transfer (migration) and the locally generated excited states may migrate throughout a particle. Without wishing to be bound by theory, in this way the excitations will generally tend to transfer to lower energy states that may be introduced by either covalent of physically mixing in a structure that has a lower band gap. In some embodiments, this method may produce materials that have a large separation from the excitation and the emission that may be an advantage to measurements wherein excitation light needs to be excluded or filtered from the detection. Energy migration may occur in mixtures of materials. This may involve molecular and polymeric chromophores. The materials shown that have the large 3-dimensional pentiptycene unit in the backbone are particularly well suited to form stable compositions with other polymers or molecules as a result of the free volume promoted by structure. The materials that are shown below all have a majority portion of the composition that has a higher band gap and a minority portion of the composition that has a lower band gap. In the structures shown y>x and R has the usual meaning to one skilled in the art. An advantage of this embodiment is that the majority portion of the composition may be uniformly excited and the wavelength where the majority emission occurs is dominated by the minority portion of the composition.

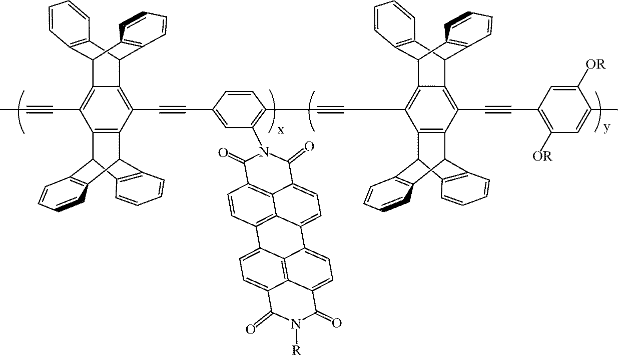

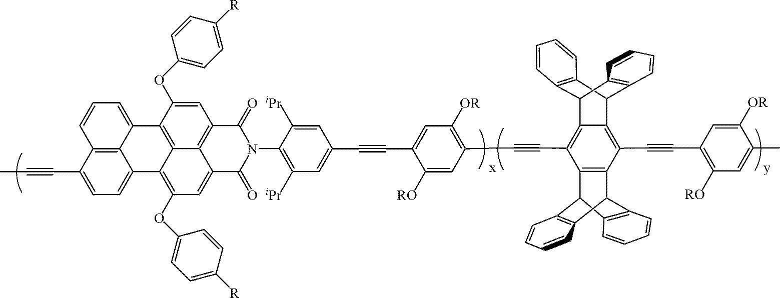

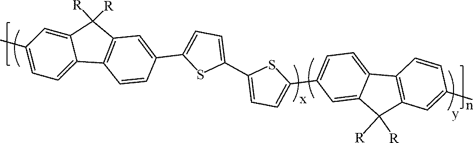

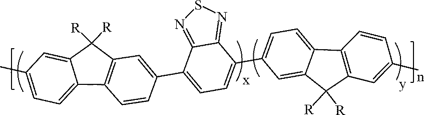

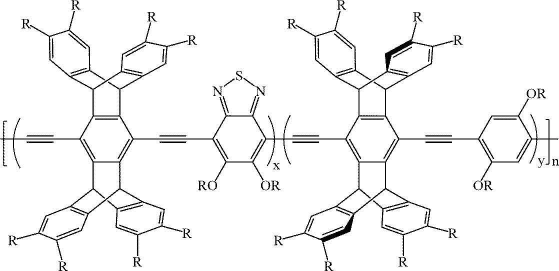

In some embodiments, it will be advantageous to make use of luminescent optical elements in the particles that undergo delayed emission. This process allows for a light to be detected after a delay from the excitation. An exemplary advantage is that other fluorescent signals may not be present after the delay. It is possible that fluorescent and delayed emission signals may be used together to provide additional information. Materials that display delayed emission include metal phosphors and organic molecules displaying thermally activated delayed fluorescence. Well known metal phosphors of particular interest are those based on gold, iridium, and platinum organometallic compounds and coordination compounds based on ruthenium, europium, and terbium. Another type of material that may undergo delayed emission are materials that have singlet and triplet states that are in thermal equilibrium. These materials undergo what is called thermally activated delayed emission (TADF) and the luminescence from these materials is delayed because of the fact that upon excitation the singlet state undergoes intersystem crossing to the triplet state which has a very long lifetime and hence very slow radiative rate. Without wishing to be bound by theory, the triplet state and singlet state thermally equilibrate and the higher radiative rate of the singlet state will generally result in emission. However, the fact that the majority of the time is spent in the triplet state, the emission lifetime is longer than would be expected by the radiative rate of the singlet.

The cores of luminescent particles, in some embodiments, will be hydrophobic and to create stable suspensions in water and afford efficient bioconjugation, surfactants and/or reactive elements are useful. In some embodiments, these surfactants are polymeric in nature and may contain reactive elements including amines, azides, carboxylates, tetrazines, trans-cyclooctenes, cyclic alkynes, maleimides, thiols, disulfides, reactive esters, or norborenes. Non-limiting examples of polymeric surfactants are shown below that may provide for bioconjugation directly of be activated to allow for bioconjugation. In some embodiments, it will be useful to use chemistry to link the polymeric surfactants with multifunctional groups or polymers. These groups may in come embodiments provide additional sites for bioconjugation or for adding groups to ensure that the particles do not display non-specific bonding to biological species, particles, or surfaces. It is also possible to use biomolecular recognition elements to functionalize particles. In some embodiments, a biotin may be attached to a particle and a functionalized streptavidin may then bind to the surface. Alternatively, streptavidin may be bound to the surface and a functionalized biotin may bind to the surface. In other embodiments, Protein A may be bound to the surface of the particle which will bind to an antibody.

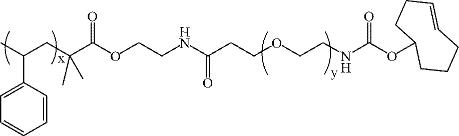

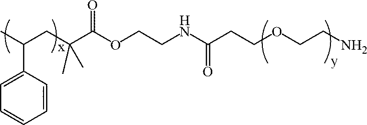

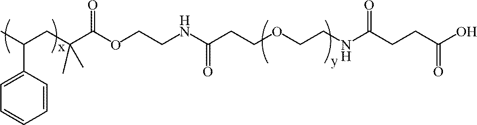

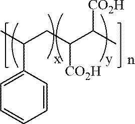

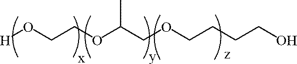

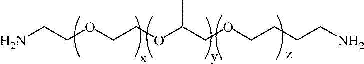

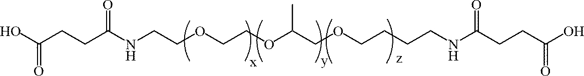

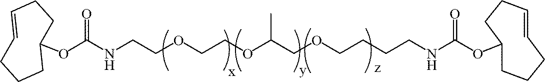

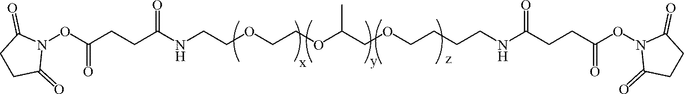

In some embodiments, reflection or scattering from particles will be used to indicate the presence of the analyte. The wavelength of light reflected or scattered by a particle may depend on the nature of the particle. It may depend on the composition and if metal particles, oxides, the angle of interfaces, or periodic structures are present. In the case of particles created from chiral nematic liquid crystals the helical pitch determines the wavelength of reflected light. The type and amount of the chiral molecules causing the twisting of the liquid crystal molecules determines the pitch and the wavelength reflected. In some embodiments, photonic block copolymers will serve as reflectors, the structures, spacing and refractive index contrast between the blocks may change the wavelengths reflected. In other embodiments, particles may reflect light as a result of curved interfaces between two different refractive index materials. Curved interfaces may be used to reflect light in this regard. In the case of reflective materials, the angle at which the active photonic elements are oriented with regard to the exciting light and the direction at which the light is detected may give rise to changes in the efficiency of the reflectivity and the wavelength. In other embodiments metal nanoparticles, such as gold nanoparticles, are used to scatter light and create an optical signal.

In some embodiments, a multiplexed assay using a mixture of different luminescent materials or reflector encoded particles with associated different receptors, may be used to detect multiple analytes at one time. Such an assay may be used to differentiate between concentrations of closely related species, or detect simultaneously multiple species. For example, in bacterial detection it could be possible to create an assay that detects within a single vessel all relevant pathogenic organisms of interest for a particular food production process. Similarly, it could be possible to have a test that simultaneously detects for influenza viruses that cause Flu A and Flu B, but also detect the coronavirus responsible for the disease COVID-19. Multiplexed assays may contain multiple encoded emissive particles with signaling entities. The recognition elements (e.g., moieties) on encoded to specific particles may similarly be diverse and non-limiting examples include antibodies, proteins, oligonucleotides, DNA, RNA, carbohydrates, glycoproteins, lectins, virus capsids, or synthetic receptors.

In some embodiments, the density of the materials that make up the emissive particles with signaling entities may be important. The emissive particles may be either more or less dense than the water phase. In some embodiments, the rate at which the particles float to the surface or drop to the bottom of a vessel is important and may affect the rate at which each measurement cycle may be performed. It is generally advantageous for measurements to be made as fast as possible. Methods to create more buoyant particles include creating porosity within the particles by generating air pockets or free volume. In some embodiments, extraction of a non-polymerized material from a polymer particle may provide pores capable of creating air pockets and/or free volume. In some embodiments, it may be important that the pores have hydrophobic character so that they do not fill with water. Incorporation of molecules that have elements such as halogens or metals may be used to create particles that have higher densities. One skilled in the art, will understand materials that pack densely or have heavier elements may produce materials with higher densities.

In some embodiments, measurements will be performed on the sidewalls of a vessel. In some embodiments, the emission will be monitored on the sidewall of the vessel. To minimize the background emission in a measurement from a point on the sidewall of a vessel, the emissive particles may either float to the top or sink to the bottom. To detect a biological species, particles may be linked (conjugated) to a recognition element (e.g., moiety). The recognition element may be considered as either a receptor or a ligand. A receptor typically binds around (partially or completely engulfs) its complementary ligand. In biological recognition, the site that the antibody binds to is generally referred to as epitope. In the more general description of biological recognition and epitope is generally referred to as a ligand, and an antibody or equivalent binding element is called a receptor. In some embodiments, ligands may be used to detect receptors and/or a species associated with a receptor. That is the receptor may be the analyte. Alternatively, receptors may be used to detect ligands and/or species having ligands. In this case the ligand or a species associated with the ligand are the analyte. For example, an antibody may be used to detect a species containing the target antigen. However, the antigen or a species having the antigen may be used to create a method to detect specific antibodies. Receptor-ligand interactions are often referred to as having lock and key interactions. This physical analogy reflects that the lock surrounds the key and that there is a special recognition between the lock and the key. In some cases, it may be or interest to have emissive particles containing signaling entities without recognition elements present as a control element. In some embodiments, the presence of these species may indicate that the solution has been prepared properly and non-specific association of these groups to the emissive particles, may indicate that there is a problem with the assay or a complexity with the sample. For an assay to recognize a particular analyte, it may be useful that at least one emissive particle contain a recognition element that will associate with the analyte. If these particles are placed in solution with the analyte, an association may take place. It is possible to have emissive particles with different recognition elements assemble around a mixture. The interactions may be polyvalent or monovalent in nature and may involve ligands as well as receptors.

In some embodiments, antibodies will be used as the receptors to bind to antigens associated with the analytes. Antibodies may be mono-clonal or poly-clonal. A monoclonal antibody will generally bind to a specific epitope, which may be a substructure of the antigen. In many cases the antigen is a protein that may be monomeric or multimeric, it may be excreted or bound to the surface of a cell, organism or surface. A polyclonal antibody is a mixture of antibodies that broadly recognize a target. These receptors may bind to multiple epitopes and protein antigens. Antibodies are large biomolecules with molecular weights typically of the range of 150,000 Daltons.

In some embodiments, it is useful to have receptors that have lower molecular weight. One type of receptor is derived from what are known as nanobodies or as single-domain antibodies that are derived from natural camelid antibodies. Generally, the nanobody structure referred to also as VHH are only a fraction of the size of an entire immunoglobulin (IgG) antibody. The smaller size of these receptors may allow for improved stability, less tendency to aggregate, and lower production costs as compared to antibodies. Other families of proteins may be used to create receptors and one class of thermal stable protein receptors are the reduced-charge Sso7d (rcSso7d) engineered proteins. Protein receptors may be identified by using selection processes from large libraries and in these methods the receptors are selected for their binding to specific antigens or structures associated with the target analytes. In some embodiments, the genes encoding the final protein receptors may be used in bioproduction of these materials. In these processes structures may be added that may allow for facile attachment (conjugation) to particles. Lectins are another class of receptors that may provide recognition and these receptors bind to carbohydrate containing molecules. Carbohydrate molecules may have complex structure and cells or organisms may be recognized by the complex carbohydrates that are attached to their surfaces. Protein nucleic acids may also be developed to bind molecules of interest. Additionally, there are many small molecule receptors that may be used to bind to analytes of interest.

In some embodiments, assay may be produced if the particles are functionalized with ligands that will bind to receptors that are associated with the analyte. In some embodiments, the ligands may be carbohydrates and the analyte may be bacteria. In other embodiments, the ligands may be carbohydrates the analyte may be a virus. In some embodiments, influenza virus may be the analyte and molecule containing a sialic acid group is used as the ligand.

In some embodiments, nuclei acids are the analytes and are also the recognition elements. A system designed to detect a specific DNA or oligonucleotide sequence may be produced provided that the luminescent particles are functionalized with sequences that may bind to the target DNA/oligonucleotide, or a hybrid complex thereof, at the same time.

In some embodiments, individual particles may have multiple recognition elements. The recognition elements may be chosen to recognize a specific analyte and multiple recognition elements may target different features of an analyte. In some embodiments, multiple recognition elements may be used to impart a broader scope of the analytes that the particles may be used to detect. In some embodiments, particles may be designed to recognize classes of bacterial or potentially all bacteria. In other embodiments, particles may be designed to recognize related classes of viruses.

In some embodiments, the functionalization of particles with recognition elements may be performed as part of the initial particle assembly. For example, the recognition element may be associated with a polymer or surfactant that assembles on the surface of the particle during the dispersion process. In some cases, the recognition element may provide surfactant characteristics that stabilize the particle during its formation. In some embodiments, it is desirable to be able to produce particles having different optical properties that may functionalized at a later stage with recognition elements. In so doing it is possible to be more efficient in creating different particles for specific applications. In some embodiments, one type of base particle may be functionalized at a late stage. In some embodiments, particles may be functionalized immediately before using in an assay. In other embodiments, particles may be functionalized in situ in the assay. Late-stage functionalization may allow for more sensitive recognition elements to be added just prior to an assay.

There are many suitable methods for covalently linking recognition elements to the surfaces of particles. These include the formation of amide linkages, thiol additions to alkenes, nucleophilic aromatic substitution, cycloaddition of organic azides to alkynes, Diels Alder reactions, photochemical reactions, condensation of aldehydes with amines, enzyme catalyzed reactions, and the like. In some cases, non-covalent methods may be used. Biotinylated particles may be bound to receptors via noncovalent assembly with streptavidin. Oligonucleotides may be used as recognition elements as well as a method to bind other elements to particles. The surface chemistries of the particles may be chosen by design to allow for the efficient attachment of receptor groups. Additionally, attachment of Protein-A to a particle may be used as a general method to non-covalently attach specific types of antibodies.

In some embodiments, the density of receptors on the surfaces of the particles may be adjusted for optimal performance. In general, it is advantageous to have a sufficient number of receptors on the surface of a particle to efficiently bind a target biological species. The interactions between the particle and the target analyte may be multivalent. A monovalent or weak interaction between a given particle and analyte, may result in binding to multiple receptors on the surface of a particle may increase the probability of a binding interaction upon an encounter of the particle and the analyte. In so doing a first point of attachment will, in some embodiments, direct a stronger binding. The presentation of the receptor on the particles surface is also often advantageous. In some embodiments, it is advantageous to have a water-soluble linkage between the receptor and the particle. Advantageously, such a water-soluble linkage may, in some embodiments, allow for the receptor to not be adversely affected by the surface of the particle, which is some embodiments will be hydrophobic. Such a tethered linkage may also allow the receptor freedom to assume geometries useful for optimal interaction with the target analyte. In some cases, the water-soluble linkage between the particle and the receptor is based on poly(ethylene glycol) or a PEG group that has integer numbers (n) of (CH₂CH₂O)ₙ between the receptor and the particle. In some embodiments, n is greater than or equal to 1, greater than or equal to 2, greater than or equal to 3, greater than or equal to 5, greater than or equal to 10, greater than or equal to 15, greater than or equal to 20, or greater than or equal to 30, greater than or equal to 50. In some embodiments, n is less than or equal to 100, less than or equal to 50, less than or equal to 30, less than or equal to 20, less than or equal to 15, less than or equal to 10, less than or equal to 5, less than or equal to 3, or less than or equal to 2. Combinations of the above-referenced ranges are also possible (e.g., greater than or equal to 1 and less than or equal to 50). Other values and ranges are also possible. Other linkages are also possible and a polymer may be anchored to a particle that has a hydrophilic region that may be used to bind multiple receptors. An example of a linkage are systems derived from block copolymers of polystyrene and polyacrylic acid. This polymeric surfactant may be functionalized by conversion of some or all of the carboxylic acid groups to reactive esters that react with amines to produce sites that may be used to link a specific receptor.

In some embodiments, the reactive groups associate with the receptor may be used to react with the surface functionality of the particles. Reactive amines in a receptor may react to form amides with reactive esters. Thiols that are attached to a receptor may be add to reactive alkenes. Carbohydrates attached to a receptor may be bound to boronic acids or oxidized in situ and reacted with amines. In many cases it is advantageous to make use of what are known as click reactions, that have bio-orthogonality. These reactions are highly specific and efficient in complex environments. A particle could be modified with a tetrazine and a receptor may be modified with a trans-cyclo-octene. These two materials may then react through a Diels Alder reaction to produce stable linkages. Alternatively, the particle may be modified with a trans-cyclo-octene and the receptor may be modified with a tetrazine to do the same process. Other click reactions involve organic azides and alkynes. Terminal alkynes may react in click reactions through a copper catalyzed process. However, copper ions are toxic and in some cases a copper free click reaction is useful wherein a cyclic strained alkyne is used and may undergo a cycloaddition reaction with an organic azide without a copper catalyst. The latter are referred to copper-free click reactions.

In some embodiments, antibodies are functionalized with multiple tetrazines that react with trans-cyclo-octene on the surfaces of the luminescent nanoparticles. In some embodiments, amines on the antibodies are reacted with active esters to attach the reactive functionality. The reagents may also be reversed wherein the trans-cyclo-octene is added to the antibody and the tetrazine is attached to the particles.

The precision of the attachments between the receptors and the particles may also be important, in some embodiments. In some cases, multiple click reactive groups may be added to a receptor without compromising its binding properties. In other cases, it may be advantageous to have a specific connection to the particle that is remote the active binding site of the receptor to produce the most effective binding to the target analyte. In some materials such as protein receptors that may be produced by recombinant methods, it is possible to encode for specific functional groups such as series of amines or thiols at a particular location of the receptor to allow for attachment to the particle of the attachment of a click reactive group. It is also possible to use highly selective enzyme reactions, for example on the carbohydrate portion of an antibody, to install functional groups. Recombinant methods may also be used to insert non-natural amino acids that have functional groups such as azides, alkenes, or alkynes, in some cases.

The methods in this disclosure may make use of a wide array or recognition elements. A particular assay may make use of one or more different recognition elements. There are embodiments, wherein a weakly associating recognition element may be paired with a stronger binding element to produce a cooperative effect.

In some embodiments, the physical manipulation of the sample vessels will be part of the analysis method. These manipulations may be done manually or with automation. In some embodiments, a mechanical arm, which may be considered as a robot, will be programed to manipulate samples for measurements. In the case of a flow channel, this may involve the act of tipping of inverting a sample. In embodiments wherein the vessels are inverted, this may also be performed by a robot. Similarly the measurements on plastic bags may also be automated with the use of robotics.

Emission measurements generally use an optical excitation and a detector. In some embodiments, the optical excitation may be performed at multiple wavelengths and the detection may be detected over a range of wavelengths with resolution of the signal. Such a device may be constructed from a commercial emission spectrometer. Simpler devices may be also produced wherein the signals are generated by a light emitting diode and the emission is detected by a photodiode. The signals may be collected in a mode wherein the emitted or reflected light is detected while exciting the sample or using delayed emission wherein the emitted light is collected after the exciting light is removed. The emitted light may also be detected as an image and this image may contain additional information that is not present in a purely intensity based measurement.

Measurements by the disclosed method may be performed directly in bags that contain samples from which the analytes are extracted. For example, a food sample may be placed in a bag and then subjected to mechanical stimulation by a stomacher. After this treatment the same bag may be interrogated by an optical system. Alternatively, analysis may be performed on samples that have been removed from an initial processing bag and added to other vessels. The samples may be fluid suspensions of analyte that may or may not contain the particles. It is possible that particles may be added and used to selectively remove analytes that bind to them from the original sample. Such a method has the advantages of enabling analysis in a more controlled medium with known characteristics. It may also allow for bacteria to be removed and placed in a growth media. Repeated measurements of bacteria growth media may provide for a measured growth curve. The initial binding and removal may also be done under conditions that promote the strongest binding of the recognition element to the analyte. The conditions may include the presence of different ions, buffers, surfactants, nutrients, or pH.

As described above and herein, methods for the detection of bacteria with limits of detection down to one colony forming unit per sample (1 CFU) by a combination of attaching high performance light producing nanoparticles (e.g., signaling entities), optimal growth media, high affinity recognition elements, efficient collection of bacteria, optimal bacteria growth conditions, highly efficient optical structures, and image analysis. The integrated system finds utility in the detection of bacterial in water, food, on surfaces, and in bodily fluids. Pathogenic bacteria can produce sever illness and potentially cause death to humans and animals. Non-pathogenic bacteria can lead to food or medical spoilage and produce other harmful produces, such as biogenic amines. General testing for all types of bacteria is also of use in determining water quality and sterility. Detection of bacteria in a rapid sensitive and inexpensive assay is critical to preventing these adverse outcomes.

Presently, robust methods for detection of trace bacteria are limited in sensitivity and also are slow relative to societal needs. For example, in commercial food production holding up shipments by waiting for test results lowers shelf life in food markets, causes logistical bottle necks, and requires production facilities to have large, often refrigerated, holding areas. The cost of individual tests can also be an impediment to broad based testing. It is often useful to understand the viability of the bacteria and their ability to multiply. In some embodiments, this can be equated to determining if bacteria are alive or dead. Detection of dead bacteria can be a useful indicator of the potential problems in the source materials, in some embodiments. Live bacteria can multiply and in many use cases the detection of live bacteria detection is the primary goal of a test.

Bacteria may be collected by a number of different methods. They can be bound to magnetic nanoparticles and then localized by the application of a magnetic field. They can be bound to nanoparticles and separated by density. They may, in some embodiments, be absorbed to surfaces having recognition elements. Alternatively, bacteria can be separated based on their size using filters with select pore sizes. In this invention, bacteria are captured by filtration and/or surface localization and are then detected by labeling with light producing nanoparticles and analyzing the optical signature. The light from the nanoparticles may come from either from photoemission or chemiluminescence, in some embodiments. Optical signals from the bacterial collected on a filter is then used to characterize the number and type of bacterial. Determination if bacteria are alive or dead can be accomplished by using recognition elements that selectivity bind only to viable (live) bacteria. Alternatively, incubation of bacteria can be used to determine growth. Bacteria are often damaged or stressed during collection or cold storage and there is often a latency for the bacteria to grow. In some embodiments inhibitors are added to a growth medium to only allow for select bacteria to multiply. If incubation and growth is used to determine if bacteria are alive or dead, low detection limits are also advantageous. Lower detection limits equate to shorter incubation times and time between sample collection and the result. Measurements can be performed after a prescribed period or periodically at shorter time intervals. The growth media can also be adjusted to allow for selected bacteria to have a competitive advantage over others in the mixture. Food samples can have many types of bacteria. For example, the native microflora of different food can present bacterial counts of 1,000,000 CFU/g or higher or nonpathogenic organisms. A successful test for pathogenic bacteria needs to be able to detect 1-3 CFUs of the target bacteria from a sample that contains as many as 1,000,000 other bacteria. In a number of embodiments, growth media are needed that allow for target bacteria to multiply efficiently in a competitive environment with other bacteria that are initially in much higher concentrations.

The claimed methods include the production of high-performance light producing nanoparticles, the chemical conjugation of biological receptors to light producing nanoparticles, the optimization of growth media, the collection of labeled bacteria, and the optical analysis of the assay. One of ordinary skill in the art, based upon the teachings of this specification, will recognize that the combination of these elements may have advantageous performance properties.

### Light Producing Nanoparticles

In some embodiments, at least two general classes of light producing nanoparticles may be useful in this invention. For example, those that are excited by light and then give of light are considered to be photo-luminescent. For example, those that are excited chemically and then give off light are considered chemiluminescent. Other classes are also possible. Generally, the light producing nanoparticles are capable of producing large optical signals that are much brighter than those associated with a single molecule dye or single molecule chemiluminescent event. The light emitting nanoparticles can be designed to produce light of different wavelengths.

Light producing nanoparticles are produced from water insoluble materials but are dispersible in water, in some embodiments. A dispersion is a state wherein particles become uniformly distributed in a solution. Stable dispersions require materials to stabilize the interface between the water insoluble materials and the water. In some embodiments, the stabilizing materials are surfactants, in some embodiments, the stabilizing materials are polymers, in some embodiments stabilizing materials are particles, in some embodiments, the stabilizing materials are biomolecules, and in other embodiments, the stabilizing materials are comprise any or all of the above materials. The size of the light producing nanoparticle size is a critical component of the claimed methods. The nanoparticles may be sufficiently small, in some embodiments, such that they can pass through filters that capture bacteria. For example, in some embodiments, if the nanoparticles are not attached to bacteria they will pass through the filters. In some embodiments, the light producing nanoparticles will be less than 1 micron in diameter. In some embodiments, the light producing nanoparticles will be less than 0.5 micron in diameter. In some embodiments, the light producing nanoparticles will be less than 0.4 micron in diameter. In some embodiments, the light producing nanoparticles will be less than 0.3 micron in diameter. In some embodiments, the light producing nanoparticles will be less than 0.2 micron in diameter. In some embodiments, the light producing nanoparticles will be less than 0.1 micron in diameter. One of ordinary skill in the art will recognize that there are different ways to create organic nanoparticles in water solutions, including but not limited to sonication, high sheer mixing, extrusion through filters, slow addition to rapidly stirred solutions, microfluidic extrusion, nucleation on seed particles, or absorption to pre-existing nanoparticles.

In some embodiments, nanoparticle size uniformity may be important. For example, larger nanoparticles that will not pass through the filters may be prevented from forming or removed. The stability of the nanoparticle dispersions may be useful, in some embodiments. For example, if the nanoparticles aggregate without the presence of a bacterial this process may, in some cases, lead to larger particles that will not pass through the filters or other collection matrices. The dispersions of particles, in some embodiments, have extended stability in solutions. In some embodiments, the dispersion stability is greater than 1 month, in other embodiments, the dispersion stability is greater than 6 months, in other embodiments, the dispersion stability is greater than 1 year. Light producing nanoparticles can be produced in water by a variety of different dispersion methods including sonication, high sheer mixing, extrusion through membranes, and microfluidics. Conditions can be developed that produce nanoparticles that meet the size limitations. In some embodiments, conjugated polymers are dissolved in a water-soluble solvent such as tetrahydrofuran and then dispersed in water containing a dispersion stabilizing material. The dispersion stabilizing material assembles at the surface of the particle and stabilizes the nanoparticles. In some embodiments, the conjugated polymer behaves as its own dispersion stabilizing material. When light emitting nanoparticles are created from tetrahydrofuran solutions, the tetrahydrofuran partitions between the water and nanoparticle phase and is then evaporated. The evaporation can be accelerated by heating and bubbling nitrogen gas through the mixture. In some embodiments, light emitting nanoparticles are produced from solutions of water immiscible solvents and in this case the solvents can evaporate after dispersion in water in the presence of dispersion stabilizing materials. In other embodiments, a solvent is retained in the final particle and this solvent can be high molecular weight and the particle can have a fluid or gel state in its core.

It is also possible that the light emitting nanoparticles are comprised of molecular dyes, dispersed emissive solids, and/or polymers. In some embodiments, stable dispersions are useful and materials to stabilize the dispersions can be used such that the nanoparticles do not aggregate and maintain a sufficiently small size to allow passage through a filter that captures bacteria.

Without wishing to be bound by theory, stability of the light producing nanoparticles is a function of the production method, the materials within the nanoparticles, and the surfactant. In some embodiments, the light producing nanoparticles do not stick together or indiscriminately attach to surfaces. In particular the light producing nanoparticles may stay well dispersed in aqueous media and should not be collected by a filter in the absence of the target bacteria. In some embodiments, polymeric surfactants can provide stability and can also be used in concert with small molecule surfactants. It will be useful, in some embodiments, that the surfactants are either bound tightly to the nanoparticles or are not toxic to bacteria. Advantageously, in some embodiments, such considerations will be useful in that bacteria will multiply as part of the assay. Exemplary polymeric surfactants include poly(styrene) with a terminal oligomeric or polymeric ethylene glycol group. Other non-limiting examples of nonionic surfactants include block copolymers of poly(ethylene glycol)/poly(propylene glycol), block polymers of poly(styrene)/polyacrylates, polysilicons with pendant ethylene glycol polymers/oligomers, Tween 20, and Triton X-100. A non-limiting example of an ionic surfactant that can also be used in some embodiments is sodium dodecyl sulfate (SDS). In some embodiments, a combination of multiple surfactants are used. Other methods for stabilizing light producing nanoparticles include attaching large biomolecules, or the physical interactions with large biomolecules. In some embodiments, bovine serum albumin (BSA) protein is added to stabilize the light emitting nanoparticles.

In some embodiments, that light producing nanoparticles are sufficiently stable that the water can be totally and partially removed without compromising the nanoparticles. In some embodiments, the light emitting nanoparticles will be stored in concentrated solutions and then added to mixtures to be used in bacterial incubation and testing. In other embodiments, the particles can be completely dehydrated either by themselves or in the presence of other materials that become part of the dried solid. In embodiments, that can be completely dried light emitting nanoparticle dispersions are created by simply adding water, buffer, and/or growth media to dry of mostly dried particles. In some embodiments, some agitation (shaking or stirring) will be needed to produce the optimal dispersions. In some embodiments, the added water will be pure distilled water, in other embodiments, the added water will contain buffers and/or growth media. The concentration process may generally, in some embodiments, allow the light producing nanoparticles to be added to a sample mixture of interest, potentially as a powder. Once the dispersion with the sample is formed the analysis can be completed. In some embodiments other materials may be added to the nanoparticles are part of the drying process. These additives can stabilize the nanoparticles or to facilitate their dispersion in water-based solutions. In some embodiments dried nanoparticles will promote longer shelf-lives of the light emitting nanoparticles than are possible in solutions. In some embodiments, the nanoparticles will be stable for more than 6 months. In some embodiments, the nanoparticles will be stable for more than 12 months. In some embodiments, the nanoparticles will be stable for more than 2 years. Stability is generally defined, as used herein, as the luminescent efficiency of the nanoparticles being at least 70% of that of the fresh as produced nanoparticles, the ability of the particles to display dispersions that will not be captured by the filter in the absence of the target bacteria, and receptors that retain their binding affinity, and selectivity for the target bacteria.

In some embodiments, the light producing nanoparticles comprise conjugated organic polymers. The term conjugated organic polymer implies that the backbone of the polymer has delocalized π-bonded electrons. The delocalized π-electron structures can comprise ring structures including phenyl rings, naphthalene rings, anthracene rings, thiophene rings, pyridyl rings, and other heterocycles. The delocalized π-electron structures can also comprise alkenes, imines, alkynes, ketones, nitriles, esters, or amides. The delocalized π-electron segments can also be interrupted by alkane segments or carbon centers that do not have π-electron states. Delocalized π-electronic systems in some embodiments, are excited by readily available light sources such as light emitting diodes, UV-flashlights, flash lamps, camera flashes, or diode lasers. A key feature of a π-electron conjugated organic polymer is that it can be very efficient at the absorption of light. In some embodiments, a given polymer can have an optical absorbance that is more than 100 times that of a small molecule chromophore. Nonlimiting examples of conjugated organic polymers that can be used to make light producing nanoparticles include backbones that comprise, polyphenylenes, poly(phenylene vinylenes), polythiophenes, poly(phenylene ethynylenes), polyfluorenes, graphene ribbons, and mixtures thereof. In some embodiments, the polymer structures shown will comprise light producing nanoparticles.

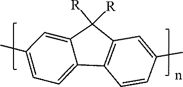

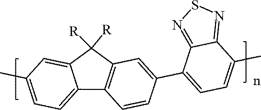

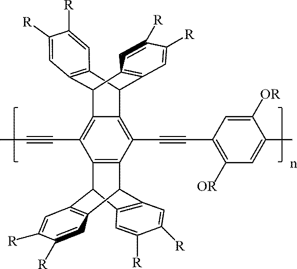

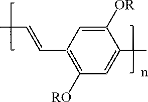

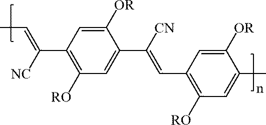

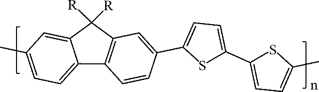

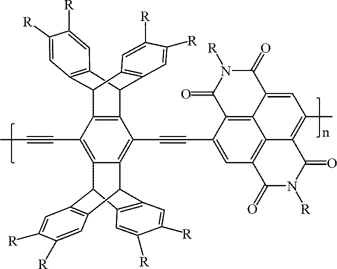

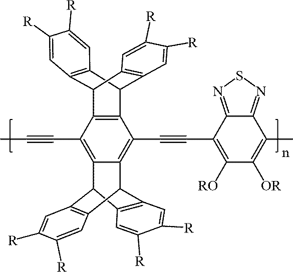

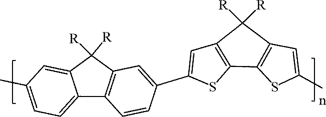

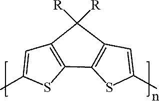

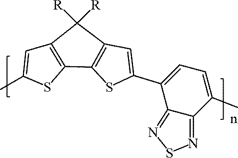

One of ordinary skill in the art will recognize, based upon the teachings of this specification, that there may be limits on the length of the polymers and that the average value of n in the chemical representation of a polymer (A)ₙ can to be more than 3, but less than 10,000. Polymers are generally mixtures of molecules with different values of n. The population molecules (distribution of n) may, in some cases, depend on the method of synthesis and the purification of the materials after synthesis. At low values of n, the polymers may have absorbance spectra that are different than those above a threshold value of n wherein the optical absorption and emission are largely independent of n. As a result, in some embodiments, it will be important to remove low molecular weight materials (small n) to create materials with more uniform optical properties. Without wishing to be bound by theory, materials with too low of n may not have uniform properties which can adversely affect their optical characteristics in light producing nanoparticles. However, too high of n may, in some cases, produce materials that have high viscosities and limited solubility, which can also complicate nanoparticle production. The conjugated polymers need not necessarily be linear and could also have branches. Branches can prevent dense packing, promote solubility, and prevent aggregation of polymer segments that can affect the emission properties. In the conjugated polymer structures shown the symbols have the regular meanings. The R groups are organic groups that can be the same or different within each example. Depending on the context, comprise alkyls, aryls, heterocycles, hydrogens, alkenes, imines, alkynes, halogens, oxygens, sulfurs, nitrogens, ketones, esters, amides, nitriles, and ring structures. R groups can be chosen to promote solubility of the materials in organic solvents. In some embodiments, some of the R groups are added to promote solubility in tetrahydrofuran. In some embodiments, some of the R groups are added to promote solubility in dichloromethane. In some embodiments, some of the R groups are added to promote solubility in chloroform. In some embodiments, some of the R groups are added to promote solubility in toluene. The R groups can also be used to provide steric barriers that prevent polymers from strongly aggregating. Aggregation can give rise to complexities in the emissive properties of the polymers and can also lead to a process wherein the polymer chains undergo what is called self-quenching. Self-quenching can lower the light emission efficiency of the polymers and in turn the light producing ability of the nanoparticles and strategic choices of R groups can be used to minimize this effect. In some embodiments, that R groups can include a polymerizable group to fix the structures in the nanoparticles. These polymerizable groups can be free radical polymerizable styrenes, acrylates or methacrylates and crosslinking monomers can be added to these materials along with thermal or photo-initiators. Crosslinking monomers will, in some embodiments, have more than one reactive group capable of forming connecting linkages between multiple polymer chains.

Conjugated organic polymers are generally efficient for the transport of the energy created by the optical absorption. The excited state if often referred to as an exciton and can move throughout a given polymer and transfer to other polymers that are in the proximity. The rate of exciton migration can be faster than the rate of the radiative relaxation of the exciton to the ground state (photoluminescence). In this way the exciton can in effect visit multiple locations within a nanoparticle before relaxing with photon emission. In some embodiments, wherein the conjugated polymer has a uniform electronic structure, this exciton migration process may not be apparent and the emission profile may reflect the dominant electronic structure of the polymer. Specifically, the polymer may, in some cases, emit light at a wavelength that is defined by the polymers bulk electronic structure. However, if polymer has a heterogenous electronic structure, which can be created by adding comonomers to the polymer backbone or by including other energy accepting chromophores in the nanoparticle, then the emitted wavelength can be longer than would be expected from the bulk electronic structure of conjugated polymer. Specifically, in some embodiments, a minority chromophore can dominate the emission of the material. These minority chromophores can be added dyes that need not necessarily be bound covalently to the polymer or created by adding a comonomer into the polymer structure. The minority chromophores are, in some embodiments, less than 50% of the total chromophores. In some embodiments, to create light producing nanoparticles that have uniform optical absorption properties, but different emission properties the minority chromophores will be less than 10% of the total chromophores. Each repeating unit of a conjugated polymer is counted as a chromophore in defining the amount of minority chromophores. The energy migration in the nanoparticles causes the bulk conjugated polymer in effect behaves as an energy collector (antenna) for optical energy, which is then transported by energy migration to the minority sites. This process produces a shift in the emission to longer wavelength from the pure polymer, when the exciton diffusing through the nanoparticle encounters a chromophore having an electronic state of lower than that of the bulk conjugated polymer. It can then transfer its energy to that new state. If the lower energy state is isolated such that only higher energy conjugated polymer states are around it, the exciton is trapped at the chromophore. An advantage of the selective emission from minority emitters within a light producing nanoparticle, is that the bulk electronic structure of the π-conjugated polymers can provide for uniform excitation among a diversity of nanoparticles that produce different emission characteristics. These features are useful for multiplexed assays or to ensure that the excitation characteristics do not need, in some embodiments, to be adjusted between assays having different light producing nanoparticles. This trapping can result in selective emission from the chromophore. The added minority chromophores can be covalently connected within the conjugated polymer backbone, can be a second conjugated polymer, can be physically mixed into the nanoparticles, or can be attached as a pendant group through a sidechain.

Examples of conjugated organic polymers that are copolymers containing minority chromophores are shown here. In some embodiments, the polymers are statistical copolymers, wherein the segments are mixed randomly as a result of the synthesis. The (chromophore)ₓ is the chromophore with the longer wavelength emission and the (chromophore)_{y} is the chromophore with the higher energy emission.

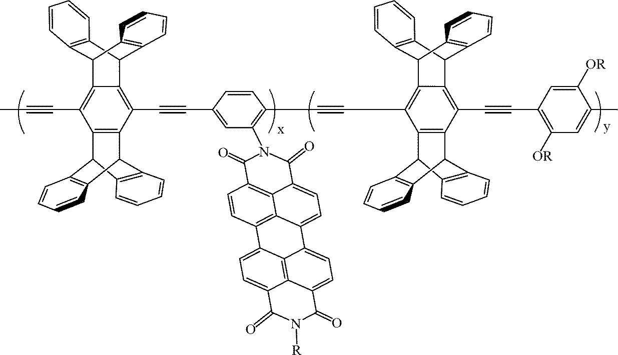

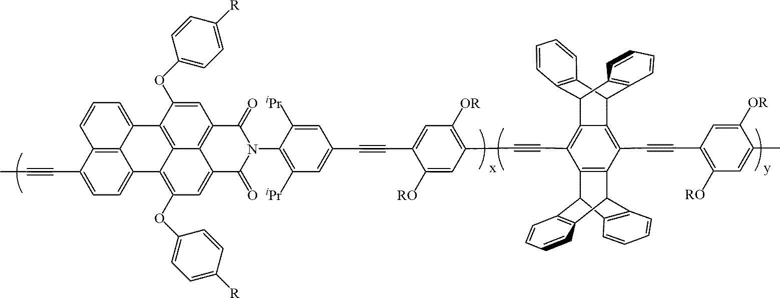

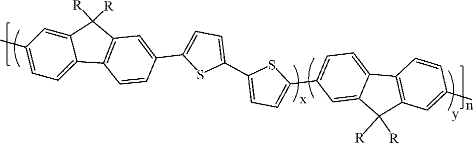

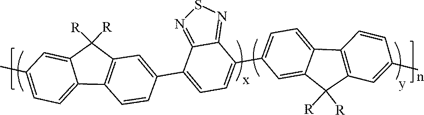

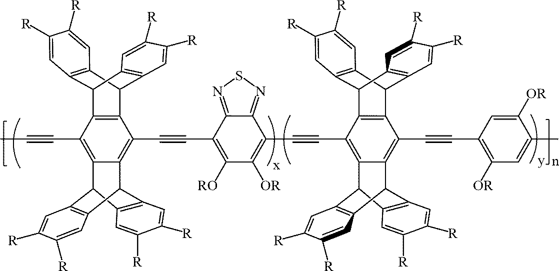

The values of x and y represent the fractional amounts of the repeating structures within the copolymers and x + y = 1. In some embodiments y > x to allow for the polymer segments with the larger band gap to be the dominate species and define the bulk electronic structure of the nanoparticles. In some embodiments x is less than (0.5)y, in some embodiments x is less than or equal to (0.2)y, in some embodiments x is less than or equal to (0.1)y, in some embodiments x is less than or equal to (0.05)y. One of ordinary skill in the art will recognize, based upon the teachings of this specification, that there may be limits on the length of the polymers and that the average value of n is generally, in some embodiments, more than 3 but will less than 10,000. Polymers are generally mixtures of molecules with different values of n. Without wishing to be bound by theory, materials with too low of a n may not have uniform properties which adversely affect their optical characteristics in light producing nanoparticles. Too high of n may, in some cases, produce materials that have high viscosities and limited solubility, which can also complicate nanoparticle production. In the conjugated copolymer structures show the symbols have the regular meanings. The R groups are organic groups that can be the same or different within each example. Depending on the context, comprise alkyls, aryls, heterocycles, hydrogens, alkenes, imines, alkynes, halogens, oxygens, sulfurs, nitrogens, esters, ketones, amides, and ring structures. R groups can be chosen to promote solubility of the materials in organic solvents or to promote different optical characteristics. In some embodiments, some of the R groups are added to promote solubility in tetrahydrofuran. In some embodiments, some of the R groups are added to promote solubility in dichloromethane. In some embodiments, some of the R groups are added to promote solubility in chloroform. In some embodiments, some of the R groups are added to promote solubility in toluene. The R groups can also be used to provide steric barriers that prevent polymers from strongly aggregating. Aggregation can give rise to complexities in the emissive properties of the polymers and can also lead to a process wherein the polymer chains undergo what is called self-quenching. Self-quenching can lower the light emission efficiency of the polymers in the light producing nanoparticles and strategic choice of R groups can be used to minimize this effect. In some embodiments, that R groups can include a polymerizable group to fix the structures in the nanoparticles. These polymerizable groups can be free radical polymerizable styrenes, acrylates or methacrylates and crosslinking monomers can be added to these materials along with thermal or photo-initiators. Crosslinking monomers have multiple reactive sites that form connecting linkages between multiple polymer chains.

Although conjugated organic polymers are particularly efficient at the transport of excitons, energy transfer is possible between small molecule organic dyes that can be placed into a particle. In some embodiments, it is advantageous to have polymers comprising localized organic chromophores. The chromophores, also referred to as emissive dyes, can be small molecules, attached to a polymer mainchain, or attached to a polymer sidechain. A single type of emissive dye can be assembled into a light generating nanoparticles, but it is also possible that the nanoparticles can be mixtures of emissive dyes. Multiple types of dyes can be used and in some embodiments 2 different emissive dyes comprise a nanoparticle and in other embodiments 3 or more emissive dyes comprise the nanoparticle. A nanoparticle can in addition to dyes or conjugated polymers, contain non-emissive small molecules or polymers. Non-limiting examples of the non-emissive small molecules are dioctylthalate of long chain hydrocarbons. Examples of non-emissive polymers include but are not limited to polystyrene, polyacrylates, or methacrylates. Polymers can be added or synthesized within the nanoparticle once it is formed and can be branched or crosslinked. In the case that one dye has a larger optical band gap, its excitation can be used to transfer energy to the lower energy chromophores. Nonlimiting examples of dyes that can be either mixed with conjugated polymers, nonemissive polymers or other small molecules to produce light producing nanoparticles are shown here. One of ordinary skills in the art will recognize that there are many potential examples of dyes that can be used. All that is necessary is that the dyes can be formulated with other molecules, polymers, and/or surfactants to give particles that provide stable dispersions. It is also clear that dyes can contain metals ions.

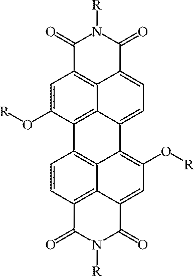

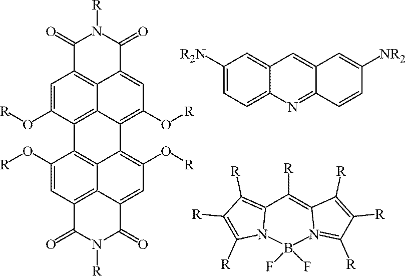

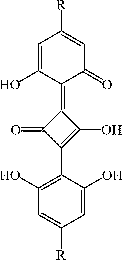

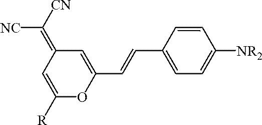

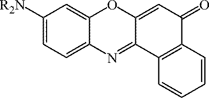

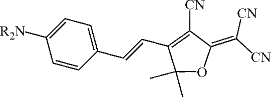

The R groups are organic groups that can be the same or different within each example. Depending on the context, these groups comprise alkyls, aryls, heterocycles, hydrogens, alkenes, imines alkynes, halogens, oxygens, sulfurs, nitrogens, ketones, esters, amides, nitriles, and ring structures. R groups can be chosen to promote solubility of the materials in organic solvents. In some embodiments, some of the R groups are added to promote solubility in tetrahydrofuran. In some embodiments, some of the R groups are added to promote solubility in dichloromethane. In some embodiments, some of the R groups are added to promote solubility in chloroform. In some embodiments, some of the R groups are added to promote solubility in toluene. The R groups can also be used to provide steric barriers that prevent dyes from strongly aggregating. Aggregation can give rise to complexities in the emissive properties of the polymers and can also lead to a process wherein the dyes undergo what is called self-quenching. Self-quenching can lower the light emission efficiency of the dyes in the light producing nanoparticles and strategic choice of R groups can be used to minimize this effect. The R groups can also be chosen to provide for compatibility with a conjugated polymer host. In some embodiments, that R groups can include a polymerizable group to fix the structures in the nanoparticles. These polymerizable groups can be free radical polymerizable, styrenes, acrylates, or methacrylates and crosslinking monomers can be added to these materials along with thermal or photo-initiators. Crosslinking monomers can interconnect dyes and/or connect dyes with polymers.

The creation of light producing nanoparticles having a shorter wavelength emitting majority chromophore and a longer wavelength emitting minority chromophore have the advantage that a family of nanoparticles can be generated that all absorb strongly at a particular wavelength. The absorption is dominated by the majority chromophore of the conjugated polymer or the majority small molecule organic chromophore. The small molecular chromophores can be assembled into polymers. Efficient excitation by a single optical source, which can be a light emitting diode, flash lamp, UV-flashlight, fluorescent light, or diode laser then uniformly excites nanoparticles that have different minority chromophores. This method is useful for multiplexed samples wherein light producing nanoparticles with a single majority chromophore can be produced that all display different emission profiles as a result of the minority chromophores that were added. The wavelength of the emitted light depends on the emission properties of the minority chromophore in the nanoparticles. In some embodiments more than 80% of the light will be emitted from a minority of chromophore. In some embodiments only 20% of the minority chromophore is needed to have more than 80% of the light emitted from the minority chromophore. In some embodiments only 10% of the minority chromophore is needed to have more than 90% of the light emitted from the minority chromophore. In some embodiments only 5% of the minority chromophore is needed to have more than 80% of the light emitted from the minority chromophore. In some embodiments less than 5% of the minority chromophore is needed to have more than 80% of the light emitted from the minority chromophore.

The ability to uniformly excite a collection of light emitting nanoparticles with one wavelength and collect signals at other wavelengths enables the ability to selectively detect multiple types of micro-organisms and/or species of bacteria in a single measurement by selectively detecting light at the different emission wavelengths. It also provides compatibility between different particles and a reader system, which can use a single excitation method but detect different wavelength emissions. In some embodiments specific nanoparticles and pair minority chromophores may be created that determine the emitted wavelength with receptors for different bacteria. In some embodiments, the colors emitted will correspond to the targeted bacteria. The uniform excitation of all of the nanoparticles, and knowledge of the efficiency of the emission from the different nanoparticles, can combine to allow for quantitative determination of the CFUs of the different bacterial in the mixture.

In some embodiments, light producing nanoparticles will display what is known as a delayed emission. Prompt emission occurs with molecules rapidly emit light after being excited. This is the most common type of emission and is generally referred to as fluorescence. An advantage of this type of emission is that it can be very efficient because the rapid rate of the emission is faster than the rates of non-emissive processes. Fluorescent materials can be very bright and have quantum yields, defined as the yield of the photos emitted from a generated excited state, that can approach 100%. Most fluorescent materials have excited state lifetimes that are less than 15 nanoseconds. Delayed emission refers to processes wherein the photons from the chromophore are delayed. The delay in some embodiments is associated with spin interconversion processes. In phosphorescence, molecules are excited and produce an electronic state known as a triplet state, which has the characteristic of having two unpaired electrons that are spin aligned. To emit a photon, a spin conversion process is needed to allow the electrons to radiatively relax to a spin paired (antiparallel spins) singlet ground state. The process of emission with spin conversion is known as phosphorescence and the excited state lifetimes can range from a microsecond to seconds. This spin interconversion causes a delay in the emission and can be facilitated by the coupling of the electronic radiative relaxation to heavy atoms like Ru, Os, Ir, Pt, Au, Ag, Re, Eu, Tb, Pb, S, Se, Br, or I.

Another way to produce delayed emission is to produce molecules that have single and triplet states that are close in energy, which allows for rapid equilibration in the excited state. The triplet states tend to be the lower energy excited states and are the dominate species. These triplets are very slow to emit light and thermal processes result in spin interconversions in the excited states that produce the singlet states that then rapidly relax. As a result, in this process known as thermally activated delayed fluorescence, the excited molecules spend the majority of their triplet states. These states are long-lived but the thermal processes cause slow interconversion to the singlet states that emit light rapidly. Typical lifetimes of efficient thermally delayed fluorescence processes range from about 1 to 15 microseconds.

Although delayed emission processes can have lower emission efficiencies than the optimal prompt fluorescence, they have the advantage that time gating of the detection can eliminate background signals. Other interfering emissive signals can limit the detection limits and accuracy of an assay. If a detector is time-gated to collect signals at a delay that is longer than 100 nanoseconds, then the majority signal will, in some embodiments, correspond to the delayed emission. To create a completely dark background, a detector may be time gated to delay collecting emission signals for a microsecond after the excitation light source is turned off. There are advantages to detecting a signal on a completely dark background that can be provided by time gating. For example, a detector gain can be set up to detect only a single light producing nanoparticle displaying delayed emission. As a result, delayed emission can provide for an enhancement in the sensitivity of an assay.

The light producing nanoparticles can also be, in some embodiments, excited chemically. These processes are called chemiluminescence and are the basis of glow sticks. In some embodiments, the nanoparticles contain at least a chromophore that can be excited by chemiluminescence. In some embodiments, the particle can also contain other reagents that participate in the chemiluminescence. By having a large number of chromophores in the light producing particle that are capable of chemiluminescence, it is possible to release a large number of photons from a single particle with a chemical stimulus. Chemiluminescence by its very nature tends to produce an emissive signal on a completely dark background in the absence of other light sources. As a result, this method if conducted in an environment that excludes background light, can give rise to very high sensitivity. The extraordinary sensitivity can be understood in the context of this invention to be the result of the light producing nanoparticles having many chromophores that can undergo the chemiluminescence reaction.

For the light producing nanoparticles to selectively attach to a bacteria of interest, receptors for bacteria specific antigens may be attached to the nanoparticle surface, in some embodiments. In some embodiments, a receptor is designed to recognize a specific type of bacteria, in some embodiments, that receptor is designed to recognize a specific bacteria that is viable, and in other embodiments, the receptor may be designed to recognize all types of bacteria. A viable bacteria is one that is capable of reproducing and a viable bacteria is considered to be alive. In some embodiments, wherein sensors are needed to confirm that sterilization has taken place or when water quality is of interest, a total bacterial count may be of interest. In some such embodiments, receptors that recognize all bacteria may be advantageous, although other receptors are possible. The receptors can be antibodies (monoclonal or polyclonal), single chain antibodies, nanobodies, lectins that bind complex carbohydrates, aryl-boronic acids, or engineered proteins. In some embodiments, that surfactants contain reactive elements that can be used in bioconjugation reactions with native functional groups attached or the receptors. For example, activated esters can be present at the surface of the light producing particle that will react with primary amines on the receptors. In some embodiments, the activated esters are esters of pentafluorophenol. In some embodiments, the activated esters are derived from a N-hydroxysuccinimide. In some embodiments, the receptors can be functionalized with a reactive element that will react with high efficiency and selectivity with groups attached to the light producing nanoparticles. These bioconjugation reactions can be designed such that they do not have any secondary reactions other than the intended conjugation reaction. In some embodiments, that reaction is known as a Diels Alder reaction and takes place between a trans-cyclo-octene and a tetrazine. The high strain of the trans-cyclo-octene in this case provides for high reaction rates, which in turn promote high efficiency of the reaction. Alternative high strain molecules include norbornene, which can also react through a Diels Alder reaction with tetrazine. Thiols can also provide for highly efficient and selective reactions. Thio-Michael reactions are useful between maleimides and thiols. In other embodiments conjugation reaction includes an organic azide reacted with a terminal or strained cyclic alkyne in a what is known as a dipolar cycloaddition reaction.

Antibodies can be functionalized chemically or enzymatically to attach functional groups that can then participate in bioconjugation reactions. For example, an organic azide can be attached to the antibody and it can be attached to a strained cyclic alkyne that is pendant to the light producing particle for a dipolar cycloaddition based bioconjugation. Alternatively, an antibody can be functionalized with one or more tetrazine groups and then reacted with a light producing particle containing a trans-cyclo-octene molecule for Diels Alder bioconjugation. The carbohydrates on antibodies can also be oxidized to create aldehydes that react with amines to allow for functionalization. One of ordinary skill it the art will recognize that the bioconjugation of antibodies to molecules, surfaces, and particles is an established practice and that there are a number of procedures that can provide similar performance. In other embodiments, an engineered protein receptor can have a single thiol for bioconjugation to a light producing particle that has pendant maleimide groups. In other embodiments, amines from a biomolecule can be reacted with a reactive ester to attach a functional group for bioconjugation. In other embodiments, a non-natural amino acid can be added to engineered protein receptor that allows for a selective reaction. In some embodiments, the non-natural amino acid contains an azide, in some embodiments, the non-natural amnio acid contains an acetylene, and in some embodiments, the non-natural amino acid contains an alkene.

The reactive groups attached to the light producing nanoparticles can be added to the hydrophilic groups associated with a surfactant or a material designed to stabilize the nanoparticle dispersion. What is key is that the reactive groups are present at the interface with water. In some embodiments hydrophilic groups that link the receptor to the particle are comprise ethylene glycol groups, in some embodiments, the hydrophilic groups comprise a zwitterionic material, in some embodiments, the hydrophilic groups comprise a biomolecule. In some embodiments, the linking groups comprise a polymer. In some embodiments, the receptor reacts with a functional poly(ethylene glycol), functional oligo(ethylene glycol), functional carbohydrate, functional poly(acrylate) or functional poly(methacrylate) that both provides for hydrophilic character of the surfactant and is also comprises one or more reactive groups. In some embodiments, the reactive groups are attached to the hydrophilic portion of a polymer surfactant. In some embodiments, the polymer surfactant is a block polymer containing a hydrophobic styrene block and a block comprising ethylene glycol. In some embodiments, that block copolymer surfactant has a hydrophobic polystyrene block and hydrophilic block derived from polyacrylic acid. Nonlimiting examples of polystyrene containing materials that can be used as reactive surfactants are the shown. One of ordinary skill in the art will understand that there will be practical limits to the average values of x and y. In some embodiments, values of x can range from 8-1000 and there will be a statistical distribution or the length of the polystyrene groups that is determined by the synthetic conditions. In the case of the ethylene glycol groups, the y value may a single number (monodisperse) or an average depending on the production method and if monodisperse materials are used in the synthesis. In some embodiments, the value of y can range from 2-600 for the ethylene glycol groups. For the acrylate derived block, x will represent an average and the average values of x range, in some embodiments, from 8-1000.

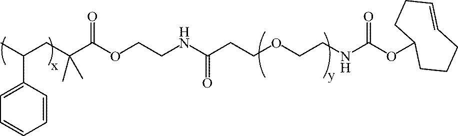

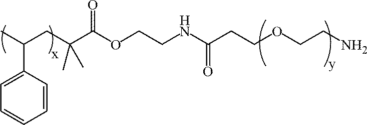

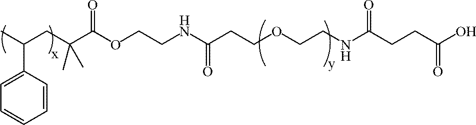

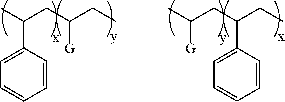

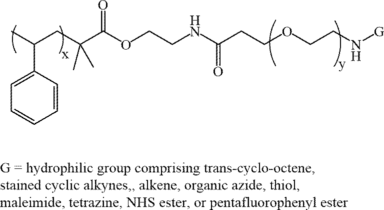

Other polymer architectures of interest are those wherein having a hydrophilic backbone with hydrophilic sites for receptor attachment positioned along the polymer backbone. Examples of polymeric surfactants with this structure are the following:

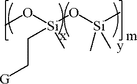

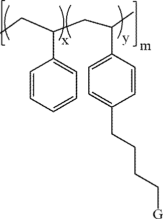

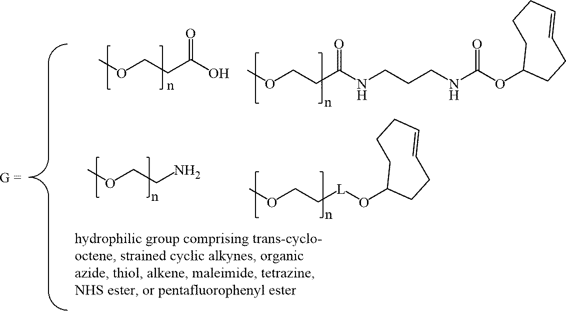

There are many other possibilities for the creation of reactive surfactants to attach receptors to the light producing nanoparticles. The reactive surfactants can have polymeric structures or be defined small molecules. Non-limiting examples of other reactive surfactant molecules are given below. The values of x and y can either be the ratio of the monomers in the case of a copolymer or relate to the length of the respective blocks in the block polymer and in this case can have values from 8 to 1000.

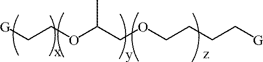

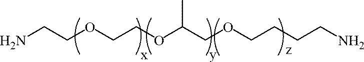

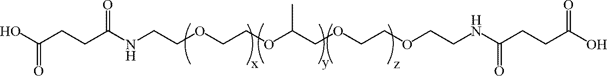

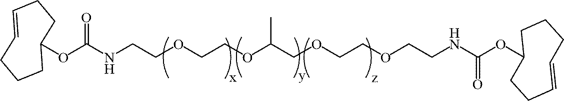

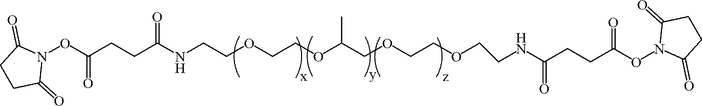

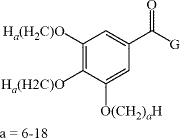

G = hydrophilic group comprising trans-cyclo-octene, strained cyclic alkynes, organic azide, thiol, alkene, maleimide, tetrazine, NHS ester, or pentafluorophenyl ester

In some embodiments, the dyes or conjugated polymers can comprise hydrophilic groups that in some embodiments can have reactive elements are capable of reacting in bioconjugation reactions. In some embodiments, all of the dyes and conjugated polymers have these elements and in other embodiments only some of the dyes and conjugated polymers have hydrophilic groups. The hydrophilic groups may be used to stabilize the dispersions and avoid the need, for example, for additional stabilizing materials. In some embodiments, the hydrophilic groups of the dyes and conjugated polymers comprising reactive groups for bioconjugation will also avoid the need for additional reactive groups to be added for efficient bioconjugation.

The detection of bacteria by binding light producing nanoparticles to the surface of bacteria is dependent upon the recognition element. In some embodiments, highly specific recognition elements are desired that detect a specific strain of bacteria or only live bacteria of a particular kind. In some embodiments, highly specific recognition can be accomplished by attaching antibodies, single chain antibodies, nanobodies, lectins, or engineered proteins. These structures can be covalently linked to the light producing nanoparticles by direct reactions with native functionality of the recognition element or by first functionalizing the recognition element with a reactive element that can be used to selectively and efficiently react with complementary reactive groups pendant to the nanoparticles. Complementarity in the reactive groups implies that one or more groups on the receptor undergoes a very specific reaction with light producing nanoparticles. In some embodiments only a single covalent bond connects the recognition element to the nanoparticle, and in some embodiments multiple linkages are possible. For example, if there are multiple reactive primary amines associated with a receptor and the reactive groups are added using a reactive ester, such as a N-hydroxysuccinimide (NHS) ester, then multiple reactive elements can be added to the antibody. However, one in ordinary skill will recognize that excessive functionalization of a receptor can lead to reductions in its ability to bind to its target antigen. In some embodiments, one or more tetrazines connected to NHS esters can be used to covalently modify a receptor. In other embodiments, one or more trans-cyclo-octene groups connected to NHS esters can be used to covalently modify a receptor. In other embodiments one or more cyclo-alkyne groups connected to NHS esters can be used to modify a receptor. In other embodiments, one or more organic azides connected to NHS esters can be used to modify a receptor. In some embodiments, enzymes can be used to modify the receptors to allow for the introduction of reactive functionality. For example, enzymes can be used to modify carbohydrates attached to an antibody in a way that allows for the attachment of a single organic azide. The methods and optimal number of groups attached to a receptor may, in some cases, vary depending upon the specific chemistry and receptor. One skilled in the art will recognize that the conjugation of the recognition element to the particles can involve other methods. The only limitation is that the conditions used for the conjugation do not degrade the ability of the recognition element to bind to the target antigens, or the stability of the light producing nanoparticle, such that an assay for detecting bacteria using the materials produced is ineffective. Ideally the attachment (bioconjugation) sites are located at locations on the receptor that are not proximate to the site that binds to the target to allow for the receptor to display its optimal binding properties.

In some embodiments, it will be important to detect all types of bacteria. These determinations can inform on suitability of water supplies for consumption by humans or livestock or to determine if conditions are completely microbe free. In these embodiments specific binding to single bacteria is using selective receptors is not optimal. In some embodiments, the detection of all microorganisms is desired. Virtually all microorganisms have carbohydrate groups on their surfaces and by using aryl boronic acids that bind to the alcohols associated with the carbohydrates it is possible to label all cellular materials with light producing nanoparticles. The interactions between boronic acid groups and carbohydrates are reversible and lacks the high binding affinity of a specific antibody for a target antigen. Multivalent interactions involving a number of boronic acid groups can provide for strong binding between the light producing nanoparticles and microbes. A nonlimiting example of a surfactant structure capable of displaying these types of interactions is shown. The boronic acid group in this structure is both the hydrophilic component of the surfactant polymer as well as the group that binds to any cellular material. This material can be derived from block copolymers of polystyrene and polyacrylic acid with post polymerization addition of the boronic acid through a standard activated NHS protocol. The values of x and y indicate the average length of the different blocks and can range from 8 to 1000. One skilled in the art will recognize that other boronic acid groups, in some embodiments, also generally bind to carbohydrates.

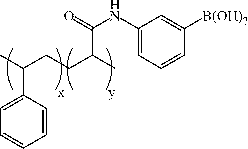

Growth media can result to accelerated growth of selected bacteria in the presence of many other bacteria that can be present in much higher levels. The growth media feeds the bacteria, but in some embodiments, it is advantageous to selectively inhibit the other background bacteria relative to the bacteria that is targeted for detection. Facilitating rapid growth of target bacterial during incubation can accelerate the ability to make live vs. dead determinations and lead to more rapid bacterial detection. Careful balancing of the inhibitors may lead to conditions wherein a bacteria of interest will flourish and actually grow at higher rate than what is possible without other bacterial present. In some embodiments, the rate growth of the target bacterial will be more than 1,000-fold enhanced of background bacteria. In some embodiments, that rate of growth of the target bacterial will be more than 10,000-fold enhanced of background bacteria.

There are a variety of different sampling methods and materials present depending on use case of the claimed bacterial detection systems. Environmental testing can involve using swabs or sponges to collect bacteria from surfaces to ensure that facilities and equipment properly sanitized. In other embodiments, bacteria may be present in a food matrix that can be shredded, crushed, and/or agitated to extract bacterial into a fluid state. Depending upon the method some filtration may be necessary to remove larger particles. Such a filtration may, in some embodiments, take care that microscopic bacteria can pass through the filter. Considering that bacteria are generally smaller than 10 microns in length, filters that prevent particles larger than 20 microns in size can be used in some embodiments. Depending on the sample, it could be advantageous to use even larger pores in the filter providing that the problematic plant/food particles do not pass through the filter. Larger pore sizes can lead to faster filtration that requires less differential pressure. In some embodiments filters with pores greater than 50 microns are preferred.

In some embodiments, the filters used to collect the bacteria have pore sizes less than one micron. Depending upon the size of the bacterial to be measured, the size of the filter’s pores can be as large as 0.8 microns. In some embodiments, to ensure that no bacteria pass through the filter the pore sizes of the filter will be 0.6 microns, in other embodiments, the filter pores will be 0.5 microns. In an exemplary set of embodiments, the lower limit of the pore size is about 0.5 microns as a result of the fact that light producing nanoparticles may pass through the filters and with smaller pore size the filtering may be slower and require higher applied pressures to force fluid through the filter. The filter materials may be inert, in some embodiments, to the light producing particles, such that they do not stick to the filters. Non-specific sticking of the light producing particles can give rise to false positive readings for bacteria. Filters can be treated to reduce or prevent sticking of the light producing nanoparticles. These treatments can be processes that chemically modify the surface of the filter, or result in the absorption of a surfactant, biomolecule or polymer to the surface.

The filters and the housing that holds the filter during the filtration process may be chosen, in some embodiments, to produce minimal background of light at the wavelengths of the light produced by the light producing nanoparticles. In embodiments where the light producing nanoparticles are excited optically, scattered light or secondary emissions from the filter or surrounding housing can produce background signals. Background light can be eliminated by using optical filters or the use of light producing nanoparticles that display a delayed emission using time gated excitation and emission collection methods to conduct analysis. In the case that the emitted light is collected under the conditions of constant excitation, filter materials and housings surrounding the measurement can be selected to minimize stray light. In particular filters that are dark materials will absorb light and reduce optical scattering. Examples of these types of filters: Sterlitech - Polycarbonate (PCTE) Membrane Filters, Gray, 0.6 Micron Part#:1270037; Sterlitech - Polycarbonate (PCTE) Membrane Filters, Black, 0.8 Micron Part#:1270062; and it4ip - ipBLACK Track Etched Membrane black polycarbonate (PC), 0.8 Micron, Part#1000M25/741N802/13. Other filters can be modified in ways to prevent background emitted or scattered light. These methods can include coating the filters with a highly absorbing non-emissive material.

Target bacteria captured on filters can be labeled with light producing nanoparticles. The nanoparticles can be excited using light to create photoluminescence or chemically to create chemiluminescence. Once samples are collected on filters, measurements can be conducted and the presence of bacteria can be indicated when collecting light from the light producing nanoparticles is detected on the filters. The measurement can be performed by collecting the total intensity of emitted light from the filter. The intensity can be used to quantify the amount of the bacteria or a threshold emission can be used to indicate the presence of bacterial. The measurement process is detailed conceptually in a nonlimiting example for a photoluminescent detection in FIG. 1B, wherein the light producing nanoparticles and bacterial are captured and then read using a fiber optic device.

In some embodiments, the imaging of the light emitting nanoparticle labeled bacteria can provide information. Multiple images may be combined, in some embodiments, into a single analysis and images can contain intensity, size and shape of the individual emissive signals, and/or the wavelength of the light emitted. Information from the image can be used to eliminate interfering signals and general background from a sample. The nature of the images may, in some cases, depend on the sample. Some samples may have larger amounts of particulate than others. In some embodiments, the bacteria counts will be sufficiently high or the bacteria become associated such that an image comprises an interconnected network of light emitting nanoparticles. The deduction of signal from background can be done in a number of ways. In one embodiment, the number of image pixels immediately proximate to each other is used to determine signal from background. For example, one or two proximate pixels can be considered to be noise, but 3-11 may be considered to be signal. Larger features are interfering particles that are too large to be bacterial or clusters of light producing nanoparticle-bacteria clusters. The clusters of pixels can be considered as islands and the size and shapes of the pixel clusters that are counted as islands can be optimized for a given bacterial assay. Once the characteristics of the island are identified which produce the best analysis, then the characteristics can be used in an assay. In some embodiments, the use of an island counting method can provide a greater than 10-fold enhancement in sensitivity over a simple prompt fluorescence intensity based assay. In some embodiments, the island counting method delivers a more than 100-fold enhancement in sensitivity over a simple prompt fluorescence intensity-based assay. Enhanced sensitivity in the detection of bacteria ensures a more reliable and faster assay. The time of an assay is the time from the introduction of the sample, which may contain only 1-10 viable bacteria, until the time wherein a result is obtained. In some embodiments, the time of an assay can be under 12 hours. In some embodiments, the time for a listeria assay capable of detecting one viable bacteria is under 12 hours.

Counting of the light emitting islands on the filter can give a quantitative determination of the amount of bacteria in addition to producing superior sensitivity. Imaging can be performed by rastering a sample with an excitation source and using a single detector or by the use of an imaging detector. Imaging devices capable of detecting light coming from objects are the basis of modern digital photography and as a result this technology is readily available. Conventional smartphones can be used to image emissions from filters. More sensitive image capture devices can be used and in some embodiments, they are used with lens structures that magnify the surface of the filter. In the case that the image is only capturing part of the entire surface area of the filter, the camera can be translated or alternatively the filter can be translated. In some embodiments, the filter will be completely imaged and in some embodiments, the filter may be imaged completely multiple times as part of an assay. In some embodiments, the collection filter containing the sample is excited with light continuously and an optical-filter is used to block the excitation light from being imaged by the image collection device. Images are best read on a dark background wherein the highest contrast of the light emission from the nanoparticles relative to background is achieved.

There are advantages provided by smart phones being used as imaging devices. One is that smartphones have pre-existing software and are connected to wireless networks. Advantageously, smartphones may be used with assays that are in the field and need not necessarily involve other equipment resources that require power. The image collection can involve the reading of a bar or QR code that provides information for the tracking of the sample, which are readily read by smartphones. Measurements can be made at remote locations and data can be transmitted for computational analysis (image processing) and provide for time and location information about the assay. In some embodiments, a desktop imaging system can be used that has higher performance optics, structures and methods for eliminating stray light, automated sample manipulation, and connection to a computer.

Imaging methods can be enhanced in some embodiments by using light producing nanoparticles that emit at different wavelengths (or combinations of wavelengths) and/or combinations particles with of prompt and delayed emission. Reference light producing nanoparticles can be added in some embodiments, to a mixture that are collected on the filter to provide a light intensity signal from which other light producing nanoparticles can be compared. In some embodiments, the reference particles could be large enough to not pass through the filter, or the reference particles could be agglutinated by an added group, or the reference particles could actively bind to a functionalized filter. In other embodiments light producing nanoparticles may be included that are designed to not interact with the bacteria or filter. In some embodiments, the particles are designed to pass through the filter and if they are detected on the filter, it will indicate that there may be interfering factors that are complicating the measurement and suggest that an additional determination is needed. In some embodiments, it may be useful for a filter or other capture surface to have recognition groups that bind and localize biological analytes for enhanced detection.

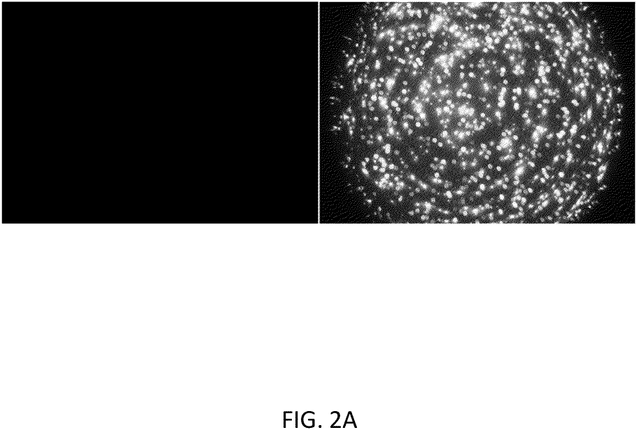

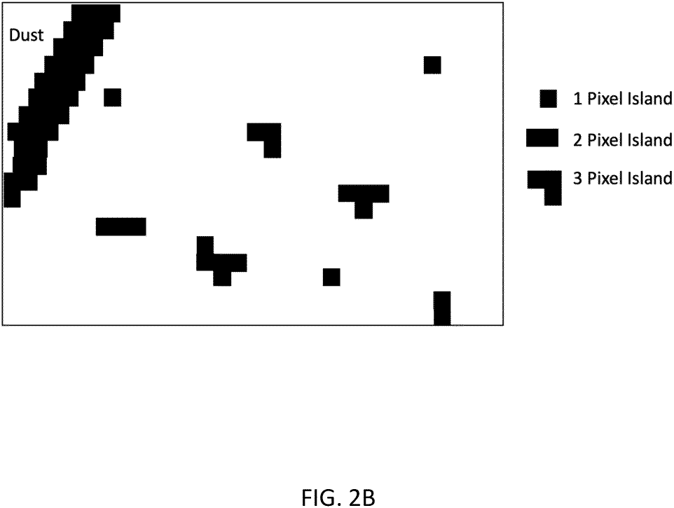

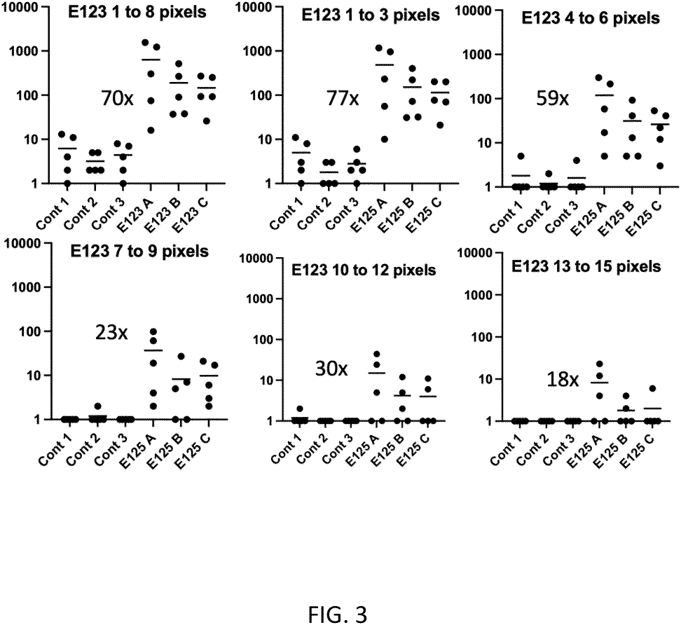

Two or more different fluorescent signals can be used with the island counting method. If light producing nanoparticles with two different colors are functionalized to bind to a specific bacteria, then the islands associated with a bacteria can contain two different emissive signatures. The absence of co-located light producing nanoparticles with two different emission signatures, can be used to discern if an island light cluster is signal or background. Islands with only one emission characteristic can be considered as background. The imaging of the bacteria collection filters in these cases can be conducted by using a detector that is capable of providing information about the wavelength of the light coming from the collection filter or by collecting multiple images that can be overlayed on each other. In some embodiments, the imaging device will take one image under one set of conditions and then take a second image under another set of conditions. In some cases, the images can be aligned so that the islands can be properly identified. In some embodiments, this can be achieved by keeping the imaging device, optics, and sample fixed in one location and changing other features. In other embodiments, it may be more effective to use indexing features to computationally be able to keep track of the spatial position of each image as it relates to the location on the collection filter. The different emission characteristics used in these methods can be based on time and a first image can be detected with prompt fluorescence and a second image can be detected under delayed fluorescence. In other embodiments, a first excitation source will selectively excite a first light producing nanoparticle and a second excitation source will selectively excite a second light producing nanoparticle. In another embodiment a signal excitation source will excite both a first and a second light producing nanoparticle and the image will be collected with a first optical filter that allows only light from the first light producing nanoparticle to reach the image detector and a second image will be collected using a second optical filter that allows only light from a second light producing nanoparticle to reach the imaging detector. In some embodiments, the criteria that define the islands that are associated with bacteria detection can be more complex than a simple number of connected pixels. For example, the spatial proximity of different distinct islands and their relative shape, size, and emission characteristics can be used. The optimal correlation of these attributes can vary with the bacteria, receptors, light producing nanoparticles, sample origin, imaging device, exposure time, light intensity, and the like. Optimized methods for particular applications can be generated by applying machine learning methods. FIGS. 2A-2B show an exemplary imaging and counting method, according to some embodiments. FIG. 3 shows data analysis for a bacterial assay showing the ability to differiate between background signals (control samples, Cont 1 and 2) and three separtate analyses with different ranges of island sizes, in an exemplary set of embodiments. The factors given are the difference in average signal relative to background and each dot represents the data collected from a given image. Multiple images may be combined to provide a larger area analysis or overlaying of images taken under different excitation or light connection consitions (wavelengths, time delays for example). In some cases these images may be analyized in multiple ways, incluing machine learning to determine the most important features to be used to determine the presence of a target biological analyte.

In some embodiments collection filter materials can be disconnected from their flow system and read using a spectrometer of other light detecting device. For real-time analysis of nonincubated samples manipulating a filter may not be a safety problem. In some embodiments, advantageously, this is because the bacterial count will be very low and with proper hygiene there are no safety issues. However, if a pathogenic bacteria sample is incubated and allowed to multiply then the same becomes hazardous and the methods may be capable, in some embodiments, of containing the sample until it can be properly disposed. As a result, in some embodiments, assays include devices designed to completely confine the samples and prevent any potential contact between an individual conducting the measurement and the samples. The initial devices may be, in some embodiments, sterilized by heating to avoid any bacterial from being in the sample chamber. Samples may, in some embodiments, then be collected and transferred in a closed system wherein incubation for specific times will occur. Filtration may, in some embodiments, then be accomplished wherein the growth medium is moved with pressure through the filter. The filter then needs to be accessible for efficient optical detection of light from excited light producing particles. A number of different devices can be imagined using microfluidic pathways, plungers, pressure, or the like to transport fluids in the system.

In one embodiment the measurement device comprises two cylinders as shown in FIG. 1C. The initial growth media is added with the sample through an inlet that has a value attached. The sample is allowed to incubate for a prescribed period and then the inner filter is pushed to the bottom and the media is force through the filter from the larger outer cylinder into the smaller inner cylinder. The filter may, in some embodiments, then be positioned at the terminus of the device proximate to an optical window that allows for optical interrogation of the filter and any bacterial that were labeled and collected. If a photoluminescence intensity or image signal is detected the filter is both excited and the emission is collected through the transparent window at the bottom of the device as shown.

In some embodiments, utilizing a chemiluminescence detection scheme, the bacterial and colocalized light producing nanoparticles collected on the filter will be excited chemically and then the emitted light will be detected. In this embodiment the optional inlet at the bottom of the device can be used to introduce the chemical reagents needed to excite the light producing nanoparticles. In some embodiments, the reagents introduced will be a hydrogen peroxide solution. The use of hydrogen peroxide as the reagent also has the added benefit that it will sterilize the inlet and prevent any dangerous emission of bacterial upon opening of the inlet.

Although not shown in FIG. 1C, the top of the device may also be closed, in some embodiments, so that the growth media will not be released. This feature is generally a redundant precaution because the filter is designed to collect the bacteria and there should be a greatly reduced bacterial load in the filtered material.

### EXAMPLES

### Formation of Light Producing Nanoparticles

A solution of poly(9,9-dioctylfluorene-alt-benzothiadiazole) (F8BT, average Mₙ ≤25000) and a suitable surfactant containing polystyrene and polyethylene oxide terminated with trans cyclooctene in THF is produced. In a first method the THF solution contains 0.48 mg/mL total solids concentration with Polymer 1 being 70% (0.34 mg/mL) of the total solids and 30% F8BT (0.14 mg/mL) of the total solids. In a second method the THF solution also contains 0.48 mg/mL with Polymer 2 having 60% (0.29 mg/mL) or the total solids and a remaining 40% of F8BT (0.19 mg/mL) makes up the remainder of the solids.

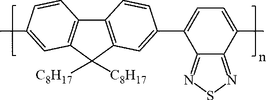

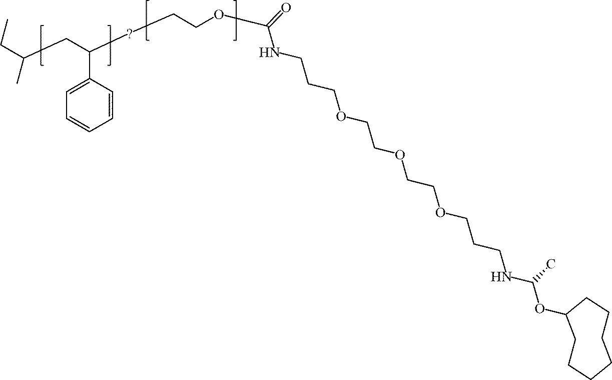

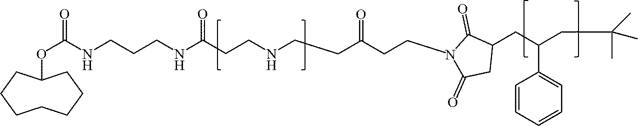

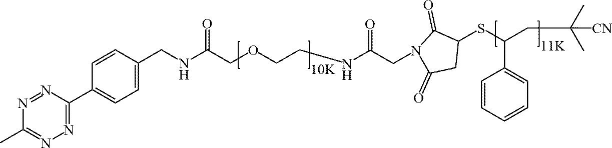

Nanoparticles are formed by using microfluidics to disperse the THF solution in water. Filtered water is placed into a Mitos P-Pump Remote Chamber 400 and pressurized (continuous phase). The THF solution is placed into a Mitos P-Pump Remote Chamber 30 pressurized (dispersed phase). Both phases are flow driven to achieve a flow rate ratio of 1.3:1 at approximately 1600 uL/min of THF solution and 1200 uL/min of aqueous phase through a 100 or 30 um Telos 2 Reagent Chip SC to produce conjugated polymer nanoparticles.

The tubing containing the dispersed polymers in water contains residual THF and is quenched into a 100 mL round bottom flask containing 10 mL of a 0.4% aqueous Tween20 solution. The round bottom flask is then equipped with a heating block and Pasteur pipette with a constant stream of nitrogen. The solution is heated at 90° C. for about 15 minutes until most of the THF has evaporated. Some evaporation of water also occurs during this process. The concentrated solution of dispersed light producing nanoparticles is removed via glass pipette and placed into a clean scintillation vial.

Nanoparticles can also be formed by an emulsification method. To this end: An ice- cold solution of polyvinyl alcohol in water (67 kDa, 0.1 to 14%, 50 g) was stirred with an IKA T25 homogenizer on 2800 rotations per minute for 15 seconds. Then a solution of F8BT (11.9 mg) and polymer 3 (27.5 mg) were dissolved in 41 g of dichloromethane was slowly added. The emulsion was stirred for 2 minutes and then transferred to the LM-20 microfluidizer. The emulsion was pumped through the reaction chamber 3 times at a pressure of 15,000 PSI and collected in a 400 mL beaker. The emulsion was placed on a magnetic stirrer and stirred at 500 RPM for 16 hours to afford the nanoparticles.

### Surfactant Synthesis

500 mg (50µMol) of Maleimide-poly(ethylene glycol)-succinimidyl [Nanosoft Polymers, order: Mal-PEG-SCM] was dissolved in 20 mL of anhydrous THF in a 100 mL round bottom flask with a magnetic stirrer. 26.3 mg (100µMol) of trans cyclooctene amine Hydrochloride [BroadPharm, order#: BP-22422] was added followed by 17.4µL (100µMol) N,N-Diisopropylethylamine. The mixture was stirred for 3 h at room temperature. A solution of 550 mg (50 µMol) of polystyrene thiol terminated [SigmaAldrich, order#:746932-1g] and 34.4 mg (120 µMol) of tris(2-carboxyethyl)phosphine hydrochloride [SigmaAldrich, order#:75259-1g] in THF (20 mL) was slowly added to the flask. The mixture was stirred over night at room temperature and poured into 300 mL of water. The suspension was then filtered over a 0.45 µm PTFE membrane [SigmaAldrich, ref#: JHWP04700] and washed with water, methanol, and hexane. The polymer was the dried under vacuum to afford 460 mg of a white solid.

Synthesis of polymer 3 surfactant started with the addition of Methyltetrazine-amine (Broadpharm; BP-22433; 23 mg) and Mal-PEG-HNS (nanosoft polymers; 2597; 500 mg) to a flask containing THF (20 mL). The solution was stirred for 2 hours and N,N-Diisopropylethylamine (Sigma-Aldrich; 75259; 17.4 µL was added. The solution was stirred for 2.5 hours.

In a separate flask, polystyrene thiol terminated (Sigma-Aldrich: 746932; 550 mg) and Tris(2-carboxyethyl)phosphine hydrochloride (Sigma-Aldrich: 75259; 34.4 mg) were added to THF (15 mL) and stirred for 2.5 hours. The two flasks were then combined and stirred over night at room temperature. The mixture was then poured into 400 mL of water. The suspension was then filtered over a 0.45 µm PTFE membrane [SigmaAldrich, ref#: JHWP04700] and washed with water, methanol, and hexane. The polymer was the dried under vacuum to afford 372 mg of a white solid.

### Conjugation of Recognition Elements to Light Producing Nanoparticles

### Functionalization of Antibodies

5 µL of a 10 mM solution of 4-Methyltetrazine-sulfo-NHS (4.3 mg/mL in DMSO) was placed at the bottom of a 1.7 mL Eppendorf tube. Anti Salmonella antibody (0.75 mg in 1 mL of PBS [abcam, order#:ab8247] was added to the tube and agitated on a Ferris wheel for 30 minutes at room temperature. 20 µL of Tris buffer (1 M of 2-Amino-2-(hydroxymethyl)propane-1,3-diol in water at pH8) [SigmaAldrich, order#:10812846001] was added and the solution was agitated for another 15 minutes at room temperature before being purified by dialysis through MWCO 20,000 Da [ThemoScientific, order#: 87735].

### Functionalization of LSP5

120 µL of a 10 mM solution of 4-Methyltetrazine-sulfo-NHS (4.3 mg/mL in DMSO) was placed at the bottom of a 5.0 mL Eppendorf tube. LSP5 (5.0 mg in 3.5 mL of PBS was added to the tube and agitated on a Ferris wheel for 30 minutes at room temperature. 200 µL of Tris buffer (1 M of 2-Amino-2-(hydroxymethyl)propane-1,3-diol in water at pH8) [SigmaAldrich, order#:10812846001] was added and the solution was agitated for another 15 minutes at room temperature before being purified by dialysis through MWCO 3500 Da [ThemoScientific, order#: 87725]. After dialysis the solution was diluted to a concentration 0.1 mg/mL LSP5 and stored in the fridge at 4° C.

### Functionalization of Light Producing Nanoparticles With LSP5

1 mL of PBS was added to 5 mL of light producing nanoparticles followed by 25 µL of 4-Methyltetrazine functionalized LSP5 (0.1 mg/mL). The vial was placed on the rocker for 2 h and then filtered through a 0.6 µM track etched membrane. The solution was then stored in the fridge until usage.

### Detection of Listeria

Samples, containing Listeria or not, were added to 5 mL Eppendorf vials, each containing 3.5 mL of sterile Brain Heart Infusion Broth (BHI) [Merk KGaA, order#: 1.10493.0500] or a growth media containing inhibitors. The samples were incubated for a set amount of time and 25 µL of the functionalized nanoparticles were added. The mixture was then incubated for another 2 hours before filtration over a black 0.8 µm membrane [it4ip, order#: 1000M25/741N802/13] in a Swinnex filter holder [Millipore, order#:SX0001300].

The sample in the filter cartridge was then analyzed by a bifurcated optical cable exciting with 460 nm light and measuring the 530 nm emission or a picture was taken through a microscope.

### Patterns in Bacterial Detection

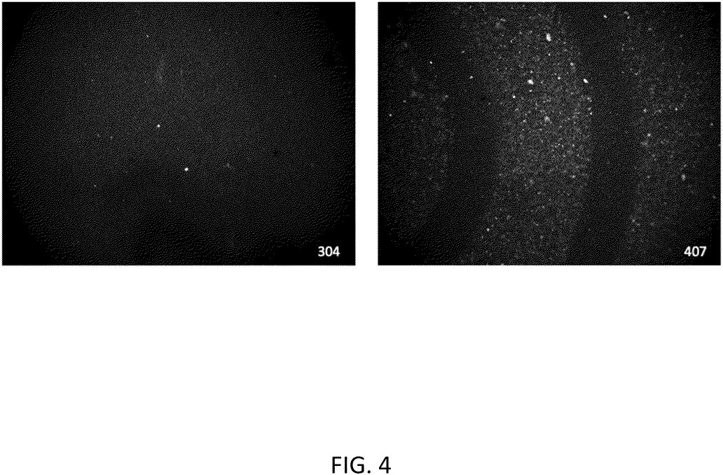

To achieve optimal detection of bacteria it is important to differentiate the presence of target bacteria relative to background samples that do not contain the target bacteria. Background signals can be, in some cases, the result of dust particles or other particles that produce a light signal or by non-specific binding of the light producing particles to surfaces other than the target bacteria. A low level of detection the total light signal from the capture surface may be similar in magnitude for samples that contain the target bacteria and samples that do not have the bacteria. However, it is possible to robustly differentiate samples that have these similar levels of signal by making use of patterns that form for the successful detection. These patterns are not present in the background signals and are produced by directed accumulation of the particles on a surface. In some embodiments, the flow of a solution through a capture material, which in preferred embodiments is a filter, can be modulated by placing restrictions at the back side (FIG. 4). In this case the restrictions can be used to create patterns wherein there is lower light signals coming from the restricted regions. In other embodiments, a capture surface can be functionalized in ways that either prevent flow by reducing wettability or by attracting bacterial by having capture recognition elements for the bacterial attached to the surface. In either case a pattern can be applied by selective deposition of different chemical or biological agents on the surface. Attaching recognition elements to a surface can also assist in achieving superior detection and it need not be limited to patterned surfaces and can be applied uniformly over an entire capture surface. Multiple levels of patterning can also be applied both by modifying the capture surface and by physically blocking flow through the capture materials.

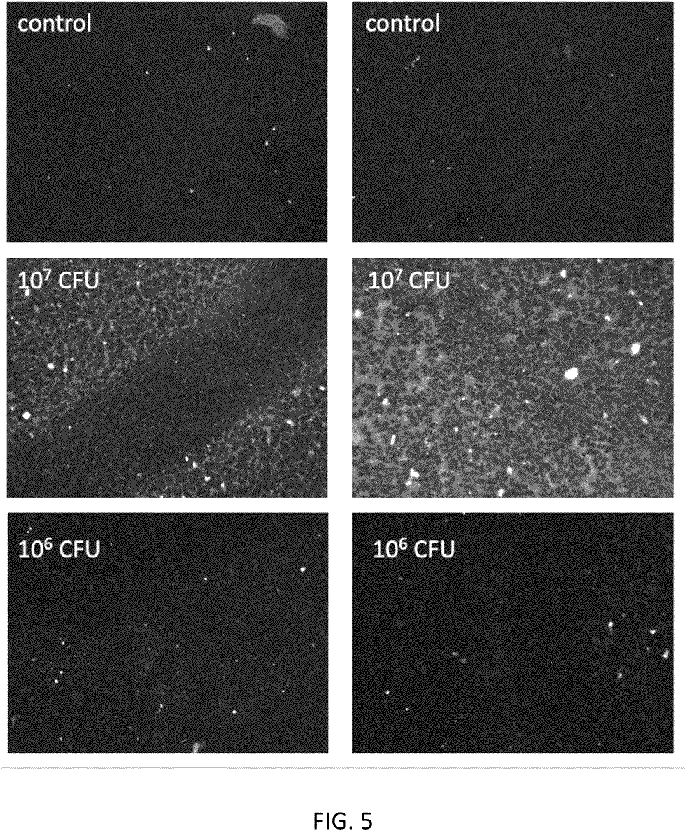

The assembly of bacteria and light producing particles also can intrinsically create patterns. This self-assembly process can lead to small structures that can have dendritic and filament characteristics. Examples of these patterns at different concentrations of bacteria are shown in FIG. 5. Generally, these are not random patterns created by non-specific binding and can may, in some cases, be used to confirm the presence of bacteria.

The pattern information produced only in the presence of target bacteria can be used to differentiate the successful detection of trace bacteria from a sample that doesn’t have the target bacteria. In some embodiments, simple visual inspection of the images will reveal the patterns and examples are shown in FIGS. 4 and 5. However, there are cases wherein patterns are non-obvious to the human brain and these patterns are only detected through computations methods such as machine learning and artificial intelligence. Encoding pattern information that differentiates between target bacteria containing samples and target bacterial-free samples can be used to create algorithms though machine learning that robustly differentiate positive from negative detection events even with very similar levels of light signals coming from a sample.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments, are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A system for monitoring a pathogenic analyte, comprising:
a reservoir configured to receive a sample suspected of including the pathogenic analyte, wherein the reservoir is configured to be essentially closed with respect to the pathogenic analyte once sealed;
a capturing surface disposed within the reservoir, configured to selectively capture the pathogenic analyte;
a sterile growth medium formulated to preferentially grow the pathogenic analyte disposed within the reservoir; the growth medium comprising:
a plurality of signaling entities comprising a moiety capable of binding to the pathogenic analyte, if present.

2. A kit, comprising:
a reservoir configured to receive a pathogenic analyte and a growth medium, wherein the reservoir is configured to be essentially closed with respect to the pathogenic analyte once closed;
a capturing surface disposed within the reservoir, configured to selectively capture the pathogenic analyte; and
a sterile growth medium formulated to preferentially grow the pathogenic analyte disposed within the reservoir; the growth medium comprising a plurality of signaling entities comprising a moiety capable of binding to the pathogenic analyte.

3. A method for monitoring growth of a pathogenic analyte, the method comprising:
introducing a sample suspected of comprising the pathogenic analyte into a reservoir;
introducing a sterile growth medium formulated to preferentially grow the pathogenic analyte into the reservoir;
closing the reservoir with respect to the pathogenic analyte;
culturing, for a desired period of time, the sample;
mixing the growth medium comprising the sample with a plurality signaling entities comprising a moiety capable of binding to the pathogenic analyte, if present;
isolating, via a capturing surface disposed within the reservoir, the pathogenic analyte; and
determining a property of the pathogenic analyte,
wherein the capturing surface is configured to selectively capture the pathogenic analyte while permitting the growth medium to pass through the capturing surface.

4. A method as in claim 3, wherein the step of determining the property of the pathogenic analyte comprises exposing the plurality of signaling entities to electromagnetic radiation and detecting, using a detector, a signal produced by the plurality of signaling entities.

5. A method as in claim 3, wherein the property is emission from a signaling unit attached to the analyte.

6. A method as in claim 3, wherein the property is the result of analyte associated reaction that provides a detectable optical change.

7. A system as in claim 1, wherein the plurality of signaling entities is a receptor dye conjugate.

8. A system as in claim 1, wherein the receptor is an antibody or recognition protein.

9. A system as in claim 1, wherein the plurality of signaling entities contains nanoparticles.

10. (canceled)

11. A system as in claim 1, wherein the plurality of signaling entities has an excited state lifetime more than 1 microsecond.

12. A system as in claim 1, wherein the plurality of signaling entities scatters light.

13. A method as in claim 3, wherein exposing, allowing, localizing, exciting, and determining the signal are conducted in a single medium.

14. A method as in claim 3, comprising determining a characteristic of the analyte in part with reference to the reference signal.

15. (canceled)

16. A system as in claim 1, wherein the particles are functionalized with a recognition element.

17-21. (canceled)

22. A system as in claim 1, wherein the analyte is selected from the group consisting of bacteria, a cell, a protein, a toxin, RNA, DNA, a virus, and an antibody.

23-33. (canceled)

34. A system as in claim 1, wherein the plurality of signaling entities comprise an emissive species containing Eu, Tb, Gd, Au, Au, Ir, Cu, Pd, Pt, Ru, Ag, Zn, or Al.

35-37. (canceled)

38. A system as in claim 1, optical signatures allow for measurement of the amount of analyte.

39-41. (canceled)

42. A system as in claim 1, the analyte is localized on the sidewall of a vessel containing a solution.

43-64. (canceled)

65-71. (canceled)

72. A system as in claim 1, wherein the bacteria detected is connected with food production, is from a water sample, or is from a subject such as an animal.

73-74. (canceled)
